(19) United States
(12) Patent Application Publication
(10) Pub. No.: US 2024/0099723 A1
(43) Pub. Date: Mar. 28, 2024

(54) INTRASACCULAR NECK BRIDGING DEVICE

(71) Applicant: Deepin Technologies, LLC, San Jose, CA (US)

(72) Inventors: Jon Schabert, San Jose, CA (US); Jiaxu Wang, San Jose, CA (US); Tiffany Tran Ngo, San Jose, CA (US)

(73) Assignee: Deepin Technologies, LLC, San Jose, CA (US)

(21) Appl. No.: 18/476,018

(22) Filed: Sep. 27, 2023

Related U.S. Application Data
(60) Provisional application No. 63/410,630, filed on Sep. 28, 2022.
(60) Provisional application No. 63/481,663, filed on Jan. 26, 2023.

(51) Int. Cl. A61B 17/12

(52) U.S. Cl. CPC A61B 17/12172; A61B 17/12113; A61B 17/12177; A61B 2017/12063; A61B 2090/3966

(57) ABSTRACT

An occlusion device includes a braided mesh body and at least one pinch member. The braided mesh body comprises plural strands each having a first end portion and a second end portion. The plural strands fold over to bring the second end portions of the plural strands adjacent to the first end portions of the plural strands forming a double-layer of the braided mesh body. In an example occlusion device, at least one of the plural strands is radiopaque, and the at least one pinch member is non-radiopaque.

### CROSS-REFERENCE TO RELATED APPLICATIONS

This application claims priority to U.S. provisional patent application No. 63/410,630 filed Sep. 28, 2022 entitled “Intrasaccular Neck Bridging Device” and U.S. provisional patent application No. 63/481,663 filed Jan. 26, 2023 entitled “Intrasaccular Neck Bridging Device,” the disclosures of all of which are hereby incorporated by reference in their entirety.

### TECHNICAL FIELD

This application relates generally to medical devices and methods of making and using medical devices. In particular, various embodiments of an intrasaccular device or occlusion device for deployment within the vasculature of a human body and a method of making and/or using the occlusion device are described.

### BACKGROUND

Occlusion devices are known and have been used in treatment of vascular disorders such as aneurysms. An aneurysm is a bulge or swelling formed on a wall of an artery in the brain or other locations of a human body. A brain aneurysm can cause severe pain, and if ruptured, lead to fetal stroke. In a non-invasive or minimally invasive treatment of aneurysms, an occlusion device may be placed in or at the aneurysm to isolate the aneurysm from blood flow, and/or, promote thrombus formation at the site. The placement of an occlusion device is typically accomplished using a delivery system, which steers the occlusion device through the vasculature of the patient to the location of the aneurysm. Once positioned at or in the aneurysm, the occlusion device is detached from the delivery system by applying electrolytic or thermal power or by activating a mechanical detachment mechanism.

One type of occlusion devices widely used is a coil system including soft, helically wound coils. Coiling has been the gold standard of care for treating aneurysms. However, the procedure, especially for treating large and giant aneurysms, often requires an excessive quantity of coils. This can extend the procedure time, resulting in greater radiation exposure to the physician, supporting staff, and patient.

Therefore, there remains a general need for medical devices for treatment of aneurysms and other vascular disorders. It would be desirable to provide an intrasaccular device which can achieve required packing of an aneurysm through implantation of a single device which can stagnate blood flow at the walls of the aneurysm to promote clotting and subsequent healing.

### SUMMARY

In one aspect, embodiments of the disclosure feature an occlusion device. In general, an embodiment of the occlusion device comprises a braided mesh body and at least one pinch member. The braided mesh body comprises plural strands each having a first end portion and a second end portion. At least one of the plural strands is radiopaque. The plural strands fold over to bring the second end portions of the plural strands adjacent to the first end portions of the plural strands, forming a double-layer of the braided mesh body. The at least one pinch member clamps the first end portions and the second end portions of the plural strands. The at least one pinch member can be non-radiopaque.

In various embodiments of the aspect, the braided mesh body has an expanded configuration in a bowl shape with a recessed portion at bottom, and the at least one pinch member rests in the recessed portion.

In various embodiments of the aspect, the occlusion device further comprises a coupler attached to the at least one pinch member. The coupler is configured to couple to the occlusion device to a delivery device. The coupler can be non-radiopaque and capable of being decoupled from the delivery device mechanically or electrolytically. At least a portion of the coupler may rest in the recessed portion.

In various embodiments of the aspect, the at least one pinch member comprises a first pinch member clamping the first end portions of the plural strands; and a second pinch member clamping the second end portions of the plural strands. In an embodiment where the braided mesh body has an expanded configuration in a bowl shape with a recessed portion at bottom, the first pinch member may rest in the recessed portion. The occlusion device may further comprise a coupler attached to the first pinch member. The coupler may be configured to couple the occlusion device to a delivery device. The coupler may be non-radiopaque and capable of being decoupled from the delivery device mechanically or electrolytically. The coupler or at least a portion of the coupler may rest in the recessed portion. In some embodiments, the second pinch member may comprise an annular ring defining a void, and at least a portion of the first pinch member may rest within the void. In some embodiments, the tips of the second end portions of the plural strands extend out of the second pinch member and are terminated with an outward inversion, forming a flowered-tip geometry of the braided mesh body.

In various embodiments of the aspect, the plural strands can be coated with an anti-thrombogenic material. In an embodiment, the plural strands can be coated with a material comprising glycosaminoglycan (Heparin) or phosphorylcholines (PC).

In one aspect, embodiments of the disclosure feature an occlusion device. In general, an embodiment of the occlusion device comprises a braided mesh body and a second end portion. The braided mesh body comprises plural strands each strand having a first end portion and a second end portion. The plural strands fold inwardly to bring second end portions of the plural strands adjacent to the first end portions, forming a double-layer of the braided mesh body. The pinch member clamps the first end portions of the plural strands. The second end portions of the plural strands are un-pinched.

In various embodiments of the aspect, at least one of the plural strands is radiopaque.

In various embodiments of the aspect, the pinch member is non-radiopaque

In various embodiments of the aspect, the tips of the second end portions of the plural strands are terminated with an outward inversion, forming a flowered-tip geometry of the braided mesh body. The flowered-tip geometry may define a void, and at least a portion of the pinch member rests within the void. In an embodiment, a coupler may be attached to the pinch member. The coupler may be configured to couple to and capable of being decoupled from a delivery device, and at least a portion of the coupler may rest within the void.

In various embodiments of the aspect, the occlusion device may further comprise a coupler attached to the pinch member, wherein the coupler is configured to couple to and capable of being decoupled from a delivery device, and is non-radiopaque.

In various embodiments of the aspect, the plural strands of the occlusion device may be coated with an anti-thrombogenic material. The coating material may comprise glycosaminoglycan (Heparin) or phosphorylcholines (PC).

In various embodiments of the aspect, the braided mesh body may have an expanded configuration generally in a bowl shape.

In an embodiment of the aspect, at least one of the plural strands is radiopaque, the pinch member is non-radiopaque, tips of the second end portions of the plural strands are terminated with an outward inversion forming a flowered-tip geometry of the braided mesh body, the braided mesh body has an expanded configuration generally in a bowl shape with a recessed portion at bottom and the un-pinched second end portions of the plural strands define a void, and at least a portion of the pinch member rests within the void.

In one aspect, embodiments of the disclosure feature an occlusion device. In general, an embodiment of the occlusion device comprises a braided mesh body and a pinch member. The braided mesh body comprises plural strands each having a first end portion and a second end portion. The plural strands fold outwardly to bring second end portions of the plural strands adjacent to the first end portions, forming a double-layer of the braided mesh body. The pinch member clamps the first end portions of the plural strands. The second end portions are un-pinched, defining an opening around the pinch member and allowing at least a portion of the pinch member to rest within a void defined at least by the opening.

In various embodiments of the aspect, the occlusion device may further comprise a ring structure, wherein the second end portions of the plural strands are attached to the ring structure.

In various embodiments of the aspect, the pinch member is non-radiopaque.

In various embodiments of the aspect, at least one of the plural strands is radiopaque.

In various embodiments of the aspect, the occlusion device may further comprise a coupler attached to the pinch member and configured to be coupled to and decoupled from a delivery device. The coupler is non-radiopaque. At least a portion of the coupler may rest within the void.

In one aspect, embodiments of the disclosure feature an occlusion device. In general, an embodiment of the occlusion device comprises a braided mesh body, a pinch member, and a flexible filler layer. The braided mesh body comprises plural strands each having a first end portion and a second end portion. The plural strands fold over to bring the second end portions of the plural strands adjacent to the first end portions forming a double-layer of the braided mesh body. The pinch member clamps the first end portions and the second end portions of the plural strands. The flexible filler layer is in between the double-layer of the braided mesh body.

In various embodiments of the aspect, the flexible filler layer may have a hole at a geometrical center of the flexible filler layer to allow the flexible filler layer to be centered at the pinch member. The flexible filler layer may be constructed from a polymeric material or a metallic foil. The the flexible filler layer may comprise two or more sections of a same or similar shape to facilitate folding of the flexible filler layer. The flexible filler layer may comprise a deployed configuration having a maximal dimension smaller than a dimension of a neck of an aneurysm to be treated. In one embodiment, the flexible filler layer may comprise a deployed configuration having a maximal dimension larger than a dimension of a neck of an aneurysm to be treated.

In various embodiments of the aspect, the pinch member may be non-radiopaque.

In various embodiments of the aspect, at least one of the plural strands may be radiopaque.

In various embodiments of the aspect, the occlusion device may further comprise a coupler configured to couple the braided mesh body to a delivery device or decouple the braided mesh body from a delivery device. The coupler may comprise a spherical body having an interference fit with the clamped second end portions of the braided mesh body, a first wire having a distal end attached to the spherical body and a proximal end attached to the pinch member, and a second wire having a distal end attached to the spherical body and a proximal end configured to couple the braided mesh body to a delivery device. The pinch member may have a slot on a side to allow the proximal end of the first wire to be pull through and fixed to the pinch member.

In one aspect, embodiments of the disclosure feature an occlusion device. In general, an embodiment of the occlusion device comprises a braided mesh body and a pinch member. The braided mesh body comprises plural strands, each strand having a first end portion and a second end portion. The plural strands fold over to bring the second end portions of the plural strands adjacent to the first end portions of the plural strands, forming a double-layer of the braided mesh body. The pinch member clamps the first end portions and the second end portions of the plural strands. The braided mesh body has an expanded angle ranging from about 30 degrees to about 135 degrees when the braided mesh body is unconstrained in an expanded configuration. In some embodiments, the braided mesh body has an expanded angle ranging from about 50 degrees to about 135 degrees when the braided mesh body is unconstrained. In some embodiments, the braided mesh body has an expanded angle ranging from about 90 degrees to about 135 degrees when the braided mesh body is unconstrained. In some embodiments, the braided mesh body has an expanded angle ranging from about 125 degrees to about 135 degrees when the braided mesh body is unconstrained.

In various embodiments of the aspect, the braided mesh body has a maximal width ranging from about 5 mm to about 15 mm when the braided mesh body is unconstrained.

In various embodiments of the aspect, the braided mesh body has a maximal width ranging from about 5 mm to about 15 mm and an expanded angle ranging from about 90 degrees to about 135 degrees when the braided mesh body is unconstrained.

In various embodiments of the aspect, the braided mesh body has a first expanded angle at a first temperature and second expanded angle at a second temperature.

In various embodiments of the aspect, the pinch member is non-radiopaque. In an embodiment, the pinch member is constructed from nitinol.

In various embodiments of the aspect, the plural strands are constructed from a material comprising a shape-memory material.

In various embodiments of the aspect, the braided mesh body or a portion of the mesh body has pores with a pore size ranging from about 20 microns to about 500 microns in the expanded configuration.

In various embodiments of the aspect, the braided mesh body or a portion of the mesh body has a porosity ranging from about 5 percent to about 95 percent in the expanded configuration.

In various embodiments of the aspect, the braided mesh body or a portion of the mesh body is coated with an anti-thrombogenic material.

In various embodiments of the aspect, the braided mesh body or a portion of the mesh body is coated with a material comprising glycosaminoglycan (Heparin) or phosphorylcholines (PC).

In another aspect, embodiments of the disclosure feature an occlusion device. The occlusion apparatus comprises: a braided mesh body comprising plural strands, each strand having a first end portion and a second end portion, the plural strands folding over to bring the second end portions of the plural strands adjacent to the first end portions of the plural strands, forming a double-layer of the braided mesh body; and at least one pinch member clamping the first end portions and the second end portions of the plural strands, wherein when the braided mesh body is unconstrained in an expanded configuration state, the inclination of the braided mesh body gradually increases and then gradually decreases from a base end closer to the at least one pinch member to a tail end away from the at least one pinch member thereof, and a ratio of a straight-line distance between the base end and the tail end of a contour line of the braided mesh body to a length of the contour line is greater than or equal to 0.8.

In various embodiments of the aspect, the ratio of the straight-line distance between the base end and the tail end of the contour line of the braided mesh body to the length of the contour line is greater than or equal to 0.9.

In various embodiments of the aspect, there is an intersection point between a connecting line between the base end and the tail end of the contour line and the contour line, and the position of the intersection point in the contour line is defined as a first position, and wherein a ratio of a width of the first position to a maximal width of the braided mesh body is greater than or equal to 0.5.

In various embodiments of the aspect, an inclination of the braided mesh body at the base end ranges from about 10 degrees to about 40 degrees.

In various embodiments of the aspect, an inclination of the braided mesh body at the tail end ranges from about 15 degrees to about 70 degrees.

In various embodiments of the aspect, the braided mesh body comprises a second position having a maximal inclination between the base end and the tail end, and wherein the inclination of the second position ranges from about 70 degrees to about 100 degrees.

In various embodiments of the aspect, a ratio of a width of the second position to a maximal width of the braided mesh body is greater than or equal to 0.5.

In various embodiments of the aspect, the maximal width of the braided mesh body ranges from 5 mm to 15 mm.

In various embodiments of the aspect, an inclination angle between the left contour line and the right contour line of the braided mesh body ranges from about 90 degrees to about 170 degrees.

In various embodiments of the aspect, the at least one pinch member is non-radiopaque.

In various embodiments of the aspect, the at least one pinch member is constructed from nitinol.

In various embodiments of the aspect, the plural strands are constructed from a material comprising a shape-memory material.

In various embodiments of the aspect, the braided mesh body or a portion of the mesh body has pores with a pore size ranging from about 20 microns to about 500 microns in the expanded configuration.

In various embodiments of the aspect, the braided mesh body or a portion of the mesh body has a porosity ranging from about 5 percent to about 95 percent in the expanded configuration.

In various embodiments of the aspect, the braided mesh body or a portion of the mesh body is coated with an anti-thrombogenic material.

In various embodiments of the aspect, the braided mesh body or a portion of the mesh body is coated with a material comprising glycosaminoglycan or phosphorylcholines (PC).

According to an aspect of the disclosure, provided is an occlusion device. The occlusion apparatus comprises: at least one pinch member; and a braided mesh body comprising a base end attached to the at least one pinch member and a tail end away from the at least one pinch member. When the braided mesh body is unconstrained in an expanded configuration state, the inclination of the braided mesh body gradually increases and then gradually decreases from a base end closer to the at least one pinch member to a tail end away from the at least one pinch member thereof, and a ratio of a straight-line distance between the base end and the tail end of a contour line of the braided mesh body to a length of the contour line is greater than or equal to 0.8. In various embodiments of the aspect, the ratio may be further set to be greater than or equal to 0.9.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a brief summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1A illustrates a side cross-sectional view of an example occlusion device in an expanded configuration according to embodiments of the disclosure.

FIG. 1B illustrates a top view of the example occlusion device shown in FIG. 1A. In FIGS. 1A and 1B, the proximal ends and the distal ends of plural braided strands of the occlusion device are clamped by a single pinch member. FIG. 1C illustrates an expanded angle (θ) of the mesh body when the occlusion device shown in FIGS. 1A-1B is unconstrained.

FIG. 2 illustrates a side cross-sectional view of an example occlusion device in an expanded configuration according to embodiments of the disclosure. In FIG. 2, the first or proximal ends of plural braided strands of the occlusion device are clamped by a first or proximal pinch member, and the second or distal ends of plural braided strands of the occlusion device are clamped by a second or distal pinch member.

FIG. 3 illustrates a side cross-sectional view of an example occlusion device in an expanded configuration according to embodiments of the disclosure. In FIG. 3, the distal end portions of plural braided strands of the occlusion device are clamped and their tips terminated with an outward inversion forming a flowered-tip geometry.

FIG. 4 illustrates a side cross-sectional view of an example occlusion device in an expanded configuration according to embodiments of the disclosure. In FIG. 4, the distal ends of plural braided strands of the occlusion device are un-pinched.

FIG. 5 illustrates a side cross-sectional view of an example occlusion device in an expanded configuration according to embodiments of the disclosure. In FIG. 5, the distal ends of plural braided strands of the occlusion device are un-pinched and inverted outwardly forming a flowered-tip geometry.

FIG. 6 illustrates a side cross-sectional view of an example occlusion device in an expanded configuration according to embodiments of the disclosure. In FIG. 6, the proximal ends and the distal ends of plural braided strands of the occlusion device are clamped by a single pinch member, and the occlusion device comprises a recessed portion at bottom to allow a detachment zone to rest within the device or to be pulled away from a parent vessel.

FIG. 7 illustrates a side cross-sectional view of an example occlusion device in an expanded configuration according to embodiments of the disclosure. In FIG. 7, the first or proximal ends of plural braided strands of the occlusion device are clamped by a first or proximal pinch member, the second or distal ends of plural braided strands of the occlusion device are clamped by a second or distal pinch member, and the occlusion device comprises a recessed portion at bottom to allow a detachment zone to be pulled adjacent to the distal pinch member or away from a parent vessel.

FIG. 8 illustrates a side cross-sectional view of an example occlusion device in an expanded configuration according to embodiments of the disclosure. In FIG. 8, the distal ends of plural braided strands of the occlusion device are un-pinched and inverted outwardly, forming a flowered-tip geometry and defining a void to allow a detachment zone to rest within the void.

FIG. 9A illustrates a side cross-sectional view of an example occlusion device in an expanded configuration according to embodiments of the disclosure. In FIG. 9A, the distal ends of plural braided strands of the occlusion device are clamped by a distal pinch member in the form of an annular ring to allow a detachment zone to rest within the void defined by the annular ring. FIG. 9B illustrates a top view of the example occlusion device shown in FIG. 9A.

FIG. 10 illustrates a method of folding an example occlusion device into a collapsed configuration for transferring into a catheter according to embodiments of the disclosure.

FIGS. 11A and 11B illustrate a method of stretching an example occlusion device into a collapsed configuration for transferring into a catheter according to embodiments of the disclosure.

FIG. 12 illustrates a method of deploying an example occlusion device in an aneurysm according to embodiments of the disclosure.

FIGS. 13A, 13B and 13C illustrate an example occlusion device in an expanded configuration according to embodiments of the disclosure. FIG. 13A illustrates a side cross-sectional view of the occlusion device in an expanded configuration. FIG. 13B illustrates an enlarged view of a portion of the occlusion device. FIG. 13C illustrates a bottom view of the occlusion device in an expanded configuration. In FIGS. 13A-13C, the proximal ends of plural braided strands of the occlusion device are un-pinched, forming an opening or hole to allow a detachment zone to recede therewithin.

FIGS. 14A and 14B illustrate an example occlusion device comprising a filler layer between the double-layer of the occlusion device according to embodiments of the disclosure. FIG. 14A illustrates a side cross-sectional view, and FIG. 14B a top view, of the example occlusion device.

FIGS. 15A-15B illustrates an example occlusion device comprising a filler layer and a detachment mechanism according to embodiments of the disclosure. FIG. 15A illustrates a side cross-sectional view of the example occlusion device. FIG. 15B illustrates an enlarged view of the detachment mechanism.

FIG. 16A and FIG. 16B illustrate a side cross-sectional view of an occlusion device in an expanded configuration according to embodiments of the disclosure. For ease of illustration, the specific construction of a braided mesh body of the occlusion device is omitted here, and merely contour lines are illustrated.

### DETAILED DESCRIPTION OF EMBODIMENTS

### Overview

Embodiments of the disclosure provide an intrasaccular device or occlusion device useful in treating aneurysms and other vascular disorders. Implantation of a single intrasaccular device of the disclosure within an aneurysm can achieve the required packing and bridging of the neck of the aneurysm to stagnate blood flow at the walls of the aneurysm and promote clotting and subsequent healing. The intrasaccular device of the disclosure may include plural braided strands that are heat-set to provide a three-dimensional (3D) expanded geometry conducive to stagnating blood flow at the walls of the aneurysm. The braided strands may have flower-shaped distal ends to form an atraumatic tip reducing the risk of damaging tissue when the device is pushed upwards in an aneurysm.

The occlusion device may include drawn filled tubing (DFT) wires or strands to provide radiopacity for visualization during delivery and deployment. The radiopaque DFT wires or strands may be clamped together by a non-radiopaque pinch member, eliminating the need for a radiopaque marker on the device which often protrudes into a parent vessel, putting the patient at risk of thromboembolic events.

The optional use of radiopaque materials in the detachment zone allows the physician to visualize delivery and deployment of the occlusion device, further reducing or eliminating the need for a radiopaque marker band on the device. The radiopaque materials may be present on the delivery system side of the detachment zone. Alternatively, or additionally, the radiopaque materials may be present on the occlusion device side of the detachment zone. By way of example, for mechanical detachment systems there are typically separate couplers on the occlusion device and delivery system respectively. Either coupler or both couplers may be constructed of radiopaque materials to improve visibility.

The occlusion device may have a recessed portion to allow the detachment zone to rest therewithin. As such, the amount of the device remained in the parent vessel can be reduced, thereby decreasing the risk of thromboembolic events. The nested detachment zone also helps concentrate the mesh body density near the center of the device, which can be especially desirable for a design where the distal braid of the device is un-pinched. The nested detachment zone may occupy the void resulting from the un-pinched braid.

The occlusion device may be applied or coated with various materials to enhance performance of the occlusion device. For example, an anti-thrombogenic coating comprising glycosaminoglycan (Heparin) or phosphorylcholines (PC) may be applied to the occlusion device to reduce thromboembolic events in case any part of the device remains in the parent vessel. A lubricious coating comprising polytetrafluoroethylene (PTFE) may be applied on the occlusion device to increase lubricity during delivery and reduce the risks of damage.

The occlusion device may comprise a double-layered mesh body including an inner braid and an outer braid. The inner braid may be clamped by a pinch member at an end whereas the outer braid is un-pinched, creating a hole or an opening to allow the inner braid to be inverted through. This design allows a detachment zone to be inherently receded or nested within the hole created, reducing or avoiding the risks of thromboembolic events which might be caused when a part of the device remains in a parent vessel.

The occlusion device may include a filler layer between the inner braid and the outer braid of a double-layered mesh body of the occlusion device. The filler layer may be constructed or configured to increase the coverage of an aneurysm neck and facilitate folding of the occlusion device when the device is collapsed or retracted into a catheter.

Example Occlusion Devices and Systems

With reference to the figures, various embodiments of an occlusion device, system, and method will be described. The figures are intended to facilitate illustration and are not necessarily drawn to scale. Certain specific details may be set forth in the figures and description to provide a thorough understanding of the disclosure. It will be apparent to one of ordinary skill in the art that some of these specific details may not be employed to practice embodiments of the disclosure. In other instances, structures, materials, components, systems, and/or operations often associated with intravascular procedures are not shown or described in detail to avoid unnecessarily obscuring description of embodiments of the disclosure.

It should be noted that while some embodiments of the disclosure are shown and described in conjunction with a procedure of treating cerebral aneurysms, the device, system, and method described herein can be configured to treat other disorders such as coronary and peripheral vascular disorders where there is an undesirable blood flow passage extending to cardiac or vascular tissue. The term “intrasaccular device” may be used interchangeably with the term “occlusion device.”

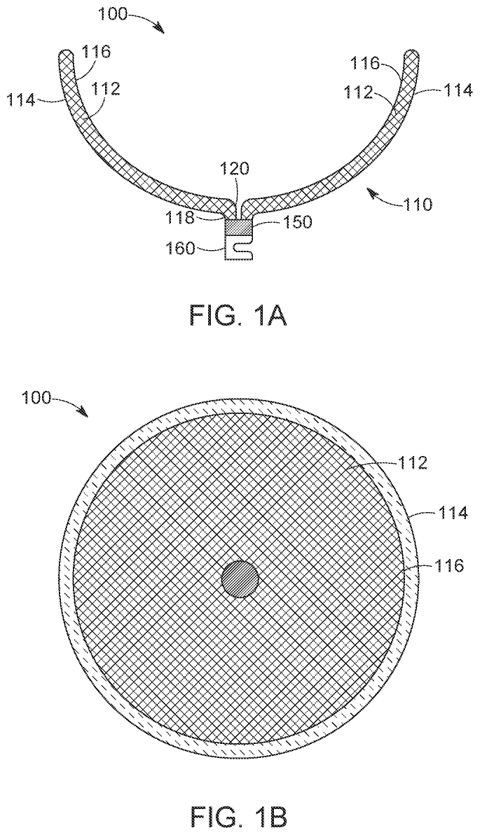

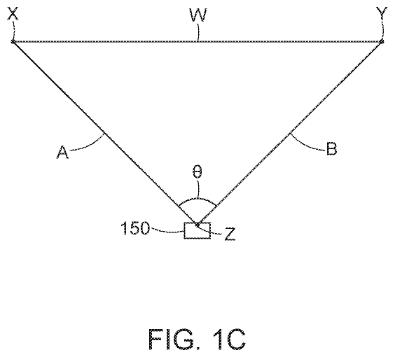

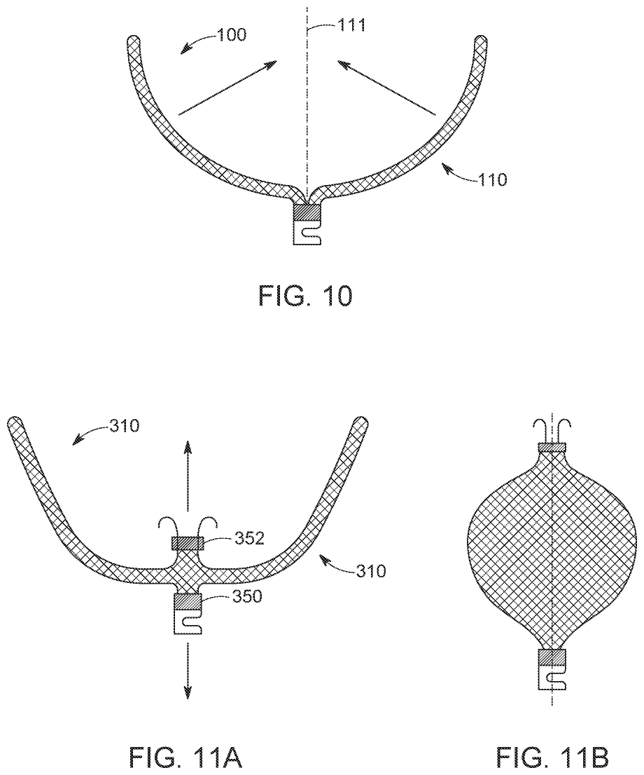

FIGS. 1A and 1B illustrate an example intrasaccular or occlusion device 100 according to embodiments of the disclosure. FIG. 1A is a side cross-sectional view and FIG. 1B a top view, depicting the occlusion device 100 in an expanded or relaxed configuration. The occlusion device 100 may also be in a collapsed configuration for transferring into a catheter for delivery, as shown in FIGS. 10-11 and will be described further below. In general, the occlusion device 100 comprises a mesh body 110 and at least one pinch member 150. The mesh body 110 may be constructed from plural strands 112. The plural strands 112 may be braided to form a braided mesh body 110. The mesh body 110 may be a double-layered mesh body, comprising a first or outer layer 114 and a second or inner layer 116 as shown. The double-layered mesh body 110 can be formed e.g., by folding the plural strands 112 over themselves to bring e.g., the distal end portions 120 of the plural strands 112 adjacent to the proximal end portions 118 of the plural strands 112. In the embodiment shown in FIGS. 1A-1B, a single pinch member 150 clamps both the proximal end portions 118 and the distal end portions 120 of the plural strands 112. The mesh body 110 or at least a portion of the mesh body 110, when in an expanded or deployed configuration, has pores with a pore size and/or porosity that can “divert” a flow of a fluid such as blood, or inhibit a blood flow through the mesh body 110 into an aneurysm or other treatment site to a degree sufficient to lead to thrombosis and healing of the aneurysm or other tissue. In general, the mesh body 110 or at least a portion of the mesh body 110 may have pores with a pore size ranging e.g., from about 20 microns to about 500 microns in the expanded configuration. By way of example, the mesh body 110 or at least a portion of the mesh body 110 may have pores with a pore size about 50-200 microns in the expanded configuration. The mesh body 110 or at least a portion of the mesh body 110 may have a porosity ranging from about 5 percent to about 95 percent in the expanded configuration. By way of example, the mesh body 110 or at least a portion of the mesh body 110 may have a porosity about 50-80 percent.

The occlusion device 100 may have a three-dimensional (3D) expanded geometry particularly useful in treating wide-necked aneurysms. By way of example, the occlusion device 100 may have an expanded configuration in a bowl or parabola shape. A wide-span 3D geometry allows a single occlusion device to bridge or seal the neck of an aneurysm, thus reducing the amount of mesh material required for filling the aneurysm, or eliminating the need for filling the entire aneurysm. When deployed, the mesh body 110 can generally conform to at least a portion of the internal wall of an aneurysm, or be in close apposition to the wall of an aneurysm to stagnate the blood flow and promote clotting and subsequent healing. It should be noted that embodiments of the occlusion device of the disclosure can be in various other 3D geometries, including e.g., spherical, cylindrical, conical, ellipsoidal, oval shapes, or any other suitable shapes, to treat aneurysms and other vascular disorders of various shapes.

With reference to FIGS. 1A-1B, the mesh body 110 may be a braided mesh body comprising plural strands 112. The strands 112 may comprise a shape-memory material, which can be metallic, polymeric, or a combination of metallic and polymeric materials. Shape-memory materials tend to have a temperature induced phase change, causing the material to have a preferred configuration or shape which can be set by heating the material above a certain transition temperature. An occlusion device “remembers” the shape set during the heat treatment and tends to assume that shape unless constrained e.g., in a catheter. Suitable metallic shape memory materials for constructing the occlusion device of the disclosure include but are not limited to alloys of nickel-titanium (NiTi) or Nitinol®, CuZnAl, FeNiAl, and so on. Suitable polymeric shape-memory materials for constructing the occlusion device of the disclosure include but are not limited to polytetrafluoroethylene (PTFE), polylactide (PLA), ethylene-vinyl acetate (EVA), and so on.

The plural strands 112 for constructing the occlusion device 100 of the disclosure may comprise strands of a single wire, filament, thread, or two or more wires, filaments or threads bundled together. According to embodiments of the disclosure, the plural strands 112 may comprise drawn filled tubing (DFT) wires, which include a core metal and an outer sheath surrounding the core metal. According to embodiments of the disclosure, the core metal of the DFT wires may comprise a radiopaque material such as platinum, gold, tantalum, tungsten, etc. The outer sheath of the DFT wires may comprise a non-radiopaque material such as Nitinol or other metal alloys. Whereas the core metal of the DFT wires provides radiopacity, the outer sheath of the DFT wires can provide shape-memory and other desirable properties such as strength, flexibility, elasticity, etc. The use of DFT wires can eliminate or reduce the need for a radiopaque marker band on the occlusion device, as will be described further below.

With reference to FIGS. 1A and 1B, the pinch member 150 clamps the end portions 118, 120 of the plural strands 112 constructing the mesh body 110. According to embodiments of the disclosure, the pinch member 150 can be constructed from a material invisible to an imaging system commonly used in the art, for example, invisible to x-ray imaging. By way of example, the pinch member can non-radiopaque. The pinch member can be constructed from a polymeric or metallic non-radiopaque material. The use of radiopaque strands or radiopaque DFT wires can eliminate the need for a radiopaque marker band or other types of markers. Conventional occlusion devices use markers such as radiopaque marker bands to help the physician visualize the devices for delivery, deployment, and monitoring. A radiopaque marker is typically made of heavy metals such as platinum, gold, tantalum, tungsten etc., which tends to protrude into a parent vessel, increasing the risks of causing undesirable thromboembolic events. Suitable non-radiopaque materials for constructing the pinch member 150 include but are not limited to: polymers such as epoxy resins, metals, or metal alloys such as nitinol, stainless steel, AgSn alloys, Pb—Sn alloys, and so on.

With reference to FIGS. 1A and 1B, the pinch member 150 can clamp the end portions 118, 120 of the plural strands 112 via various means, including bonding using a suitable adhesive such as epoxy resin, soldering, swaging, and any other suitable means known in the art. The pinch member 150 can be in any suitable form such as an annular band, ring, or collar which can secure the end portions 118, 120 of the plural strands 112.

With reference to FIGS. 1A and 1B, the occlusion device 100 may include a coupler 160 constructed or configured to couple the occlusion device 100 to a delivery system (not shown in FIG. 1A-1B) for delivery, and decouple the occlusion device 100 from the delivery system upon deployment. The coupler 160 can be attached or affixed to the pinch member 150 by any suitable means such as via bonding, soldering, or the like, and remains in the aneurysm or other treatment site after the occlusion device 100 is deployed. For ease of illustration, the coupler 160 is shown in FIG. 1A and other figures to have a hook-like feature. However, the disclosure and appended claims are not so limited. A coupler in this or other embodiments can be constructed such that an occlusion device of the disclosure can be decoupled or detached from a delivery system by applying electrolytical, thermal, or electromagnetic energy, or hydraulic pressure, as well as by actuating a mechanical system. For electrolytical detachment, the couplers on the occlusion device and the delivery system may comprise metals having different standard electrode potentials to form a junction that can be disintegrated by applying electrolytical energy. Mechanical detachment systems may include separate hooks, separate screw and thread, separate key and slot, separate ball and socket as couplers. It should be noted that while the coupler is shown in the figures as a separate piece or component for illustration purpose, the coupler can be integrated with the pinch member as a single unit.

Therefore, the term “coupler” used in the description and appended claims includes but is not limited to a particular mechanical coupling feature. The term “coupler” as used herein broadly refers to a member or element on the occlusion device which can be attached to another coupler on a delivery system, and is capable of being detached from the delivery system upon application of electrolytical, thermal, or electromagnetic energy, hydraulic pressure, or upon actuation of a mechanical system. The term “detachment zone” may be used herein to facilitate description of various embodiments of the disclosure, and refer to the combination of a coupler on the occlusion device and a complementary coupler on the delivery system. According to embodiments of the disclosure, the detachment zone may include a marker made of e.g., a radiopaque material to help the physician visualize deployment of the occlusion device. By way of example, the coupler on the delivery system side can be constructed from a radiopaque material. The use of a radiopaque material in the detachment zone can eliminate or reduce the need for a marker such as a radiopaque marker band on an occlusion device. Alternatively, or additionally, the coupler on the occlusion device side can be constructed from a radiopaque material.

With reference to FIGS. 1A and 1B, the mesh body 110 of the occlusion device 100 has an expanded configuration in a three-dimensional (3D) geometry when the mesh body 110 is unconstrained. For ease of description of various embodiments, FIG. 1C illustrates an expanded angle (θ) of the mesh body 110 when unconstrained. As used herein, the term “unconstrained” or its equivalents refer to a condition where the mesh body of an occlusion device is not compressed by a radial force e.g., unconstrained in a capillary, a packaging device, a body cavity, or is in “free air” as commonly understood by one of ordinary skill in the art. The term “expanded angle” refers to an angle formed by two lines (A and B in FIG. 1C), which shares a common endpoint (Z) at a pinch member (FIG. 1C), wherein one (e.g., line A) of the two lines extends from the common endpoint (Z) to one endpoint (X) of a maximal diameter or width (W) of the mesh body of an occlusion device when unconstrained e.g., as measured across the opening end of the mesh body, and the other line (B) extends from the common endpoint (Z) to the other endpoint (Y) of the maximal diameter or width (W).

According to embodiments of the disclosure, the mesh body 110 of the occlusion device 100 may have an expanded angle ranging from about 30 degrees to about 135 degrees. In some embodiments, the mesh body 110 of the occlusion device 100 may have an expanded angle ranging from about 50 degrees to about 135 degrees, or ranging from about 90 degrees to about 135 degrees, or ranging from about 125 degrees to about 135 degrees. In various embodiments of the disclosure, the mesh body 110 of the occlusion device 100 may have an expanded angle of 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, or any degrees therebetween. In should be noted that the above exemplary expanded angles are provided for purpose of a thorough understanding of the disclosure. The scope of the appended claims is not limited to a particular angle. Alternatively, the mesh body 110 of the occlusion device 100 may have an expanded angle smaller than 30 degrees such as 25 degrees or 20 degrees, and/or an expanded angle greater than 135 degrees such as 140 degrees, 150 degrees, 160 degrees, or 170 degrees.

In various embodiments, the mesh body 110 of the occlusion device 100 may have a width (W) ranging from about 5 mm to about 15 mm when the mesh body 110 is unconstrained. By way of example, the mesh body 110 of the occlusion device 100 may have a width of 5 mm, 7 mm, 9 mm, 11 mm, 14 mm, 15 mm, or any width therebetween. Alternatively, the mesh body 110 of the occlusion device 100 may have a width smaller than 5 mm and/or greater than 15 mm.

In various embodiments, the mesh body 110 of the occlusion device 100 may have an expanded angle ranging from about 30 degrees to about 135 degrees and a width ranging from about 5 mm to about 15 mm, or an expanded angle ranging from about 50 degrees to about 135 degrees and a width ranging from about 5 mm to about 15 mm, or an expanded angle ranging from about 90 degrees to about 135 degrees and a width ranging from about 5 mm to about 15 mm, or an expanded angle ranging from about 125 degrees to about 135 degrees and a width ranging from about 5 mm to about 15 mm. In some embodiments, the mesh body 110 of the occlusion device 100 may have an expanded angle of about 130 or 135 degrees and a width ranging from about 5 mm to about 15 mm, or an expanded angle of about 120 or 125 degrees and a width ranging from about 5 mm to about 15 mm, or an expanded angle of about 100 or 105 degrees and a width ranging from about 5 mm to about 15 mm, or an expanded angle of about 90 or 95 degrees and a width ranging from about 5 mm to about 15 mm.

Depending on the shape and/or size of the aneurysm or other vessel disorders to be treated, the occlusion device 100 may be selected to have an expanded angle and/or size suitable for the treatment. For example, a greater expanded angle may be desirable for treatment of an aneurysm having a wider neck. A smaller expanded angle may be desirable for treatment of an aneurysm having a deeper interior or for treatment of other vessel disorders. A smaller expanded angle can improve the deliverability of the device. With a smaller expanded angle, it is possible to archive a more gradual increase in the diameter or width of the mesh body or device, which can aid in sheathing and re-sheathing of the device in delivery. In general, it is desirable to reduce frictional forces at various steps in the procedure. In addition, a smaller expanded angle may allow the device to be re-sheathed more times due to less strain on the device, allowing the user or physician to re-position the device more times when needed.

According to embodiments of the disclosure, the mesh body 110 of the occlusion device 100 may have a variable expanded angle when the mesh body 110 is unconstrained, e.g., a first expanded angle at a first temperature and a second expanded angle at a second temperature. For instance, the mesh body 110 may have a smaller expanded angle when the mesh body is unconstrained at the ambient temperature and a greater expanded angle at an elevated temperature such as 37 degrees Celsius (the normal human body temperature). By way of example, at the normal body temperature (37 degrees Celsius), the mesh body 110 of the occlusion device 100 may have an expanded angle of about 130 or 135 degrees, whereas at the ambient temperature, the mesh body 110 may have an expanded angle smaller than 130 or 135 degrees respectively when the occlusion device is unconstrained. One of the advantages of an occlusion device having a variable expanded angle is that a relatively small expanded angle at the ambient temperature allows for ease of packaging of the device into a capillary or container after manufacturing, and thus reducing the risk of damaging of the device during packaging or delivery. Once the occlusion device is deployed inside the patient such as in an aneurysm, the occlusion device would have an increased expanded angle at the elevated body temperature, which can be desirable for bridging a wider aneurysm neck. According to embodiments of the disclosure, a variable expanded angle can be achieved by constructing the mesh body from a shape-memory material such as nitinol strands or filaments. The mesh body constructed from the shape-memory material can be heat set on a suitable mold such that when the mesh body is unconstrained e.g., at the ambient temperature, the device would have a smaller expanded angle. When unconstrained at an elevated temperature, the device would have a greater expanded angle. Alternatively, or additionally, the mesh body constructed from a shape-memory material can be contained in a tubular device having a predetermined size to shape the mesh body, such that when the mesh body is removed from the tubular device or unconstrained, the device would have a smaller expanded angle at the ambient temperature. At an elevated temperature, the device would resume or have a greater expanded angle.

According to embodiments of the disclosure, the occlusion device 100 can comprise various materials to enhance the performance of the occlusion device. Polymeric and/or monomeric materials and bioactive agents can be coated on the occlusion device to provide desirable properties, including reduced thrombogenicity, lubricity, drug delivery, and so on.

According to one embodiment of the disclosure, a layer of anti-thrombogenic material may be coated on the braided mesh body 110 or at least a portion of the braided mesh body 110 of the occlusion device 100 to reduce thromboembolic events. Suitable anti-thrombogenic materials include but are not limited to naturally occurring glycosaminoglycan (Heparin), phosphorylcholines (PC) such as methacryloyloxyethyl phosphorylcholine, acryloyloxyethyl phosphorylcholine, and phosphorylcholines-based monomers. Heparin materials are commercially available e.g., from Pfizer, Inc. of New York, NY. Phosphorylcholines materials are commercially available e.g., from NOF Corporation of Tokyo, Japan. An anti-thrombogenic material can be applied to the occlusion device by various methods, including e.g., spraying, dipping, and combination thereof. The preparation of a solution of an anti-thrombogenic material and the process of application of the solution on an occlusion device are generally known and thus their detailed description is omitted herein to focus on description of various embodiments of the disclosure. Based on applications, the thickness of the layer of an anti-thrombogenic material on the occlusion device 100 can range from 1 nanometer to 2000 nanometers.

According to one embodiment of the disclosure, a layer of lubricious material may be coated on the braided mesh body 110 or at least a portion of the braided mesh body 110 of the occlusion device 100 to increase lubricity during delivery and reduce the risks of damage to the devices. Suitable lubricious materials include but are not limited to polytetrafluoroethylene (PTFE), fluorinated ethylene propylene (FEP), and other suitable polymeric materials. PTFE and FEP materials are commercially available e.g., from E. I. du Pont de Nemours and Company of Wilmington, DE. A lubricious material can be applied to the mesh body 110 by various methods, including e.g., spraying, dipping, and combination thereof. The preparation of a solution of a lubricious material and the process of application of the solution on an occlusion device are generally known and thus their detailed description is omitted herein to focus on description of various embodiments of the disclosure. Based on applications, the thickness of the layer of a lubricious material on the occlusion device 100 can range from 1 nanometer to 2000 nanometers.

According to embodiments of the disclosure, the mesh body 110 is a double-layered mesh body, comprising a first or outer layer 114 and a second or inner layer 116 as shown. In an expanded configuration, the first or outer layer 114 and the second or inner layer 116 may be spaced apart at a distance, e.g., from about 0.001 inches to about 0.040 inches. The spaced apart double-layer structure of the mesh body 110 can be advantageous in that it provides an increased surface area for a small volume of clots in the space between the layers, thereby facilitating thrombus nucleation and stabilization.

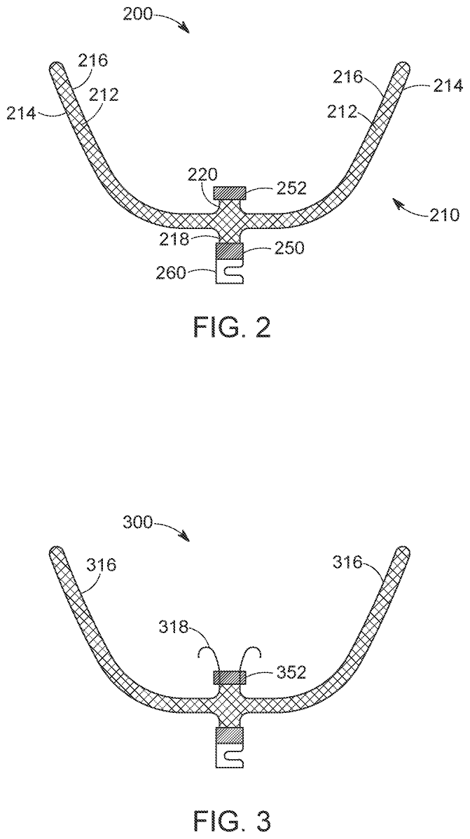

With reference now to FIG. 2, an example occlusion device 200 according to alternative embodiments of the disclosure is now described. The occlusion device 200 shown in FIG. 2 comprise a double-layered mesh body 210, including a first or outer layer 214 and a second or inner layer 216. The double-layered mesh body 210 can be formed by folding plural braided strands 212 in an initial e.g., cylindrical form over themselves along a circumferential line, bringing one ends or second ends 220 of the plural braided strands 212 adjacent to the other ends or first ends 118 of the plural braided strands. As shown in FIG. 2, the end portions 218 of the outer braid 214 can be clamped by a first or proximal pinch member 250. The end portions 220 of the inner braid 216 can be clamped by a second or distal pinch member 252. Either or both the first pinch member 250 and the second pinch member 252 can be constructed from e.g., a non-radiopaque material or a material invisible to an imaging system including such as x-ray imaging. The braided strands 212 may include radiopaque strands, such as radiopaque DFT wires. The use of radiopaque wires allows visualization of the occlusion device 200, especially at the pinch points 250, 252 where the wires 212 are bundled and clamped and the density of the radiopaque materials is greater, thereby eliminating the need for an independent or separate radiopaque marker band. As shown in FIG. 2, the occlusion device 200 may also include a coupler 260 attached to the proximal pinch member 250 for coupling to a delivery system. The coupler 260 may be constructed or configured to attached to a complementary coupler on a delivery system, and is capable of being detached mechanically or electrolytically, or in any other suitable means. The coupler 260 on the occlusion device 200 can be non-radiopaque whereas the coupler on the delivery system can be radiopaque. Alternatively, the coupler 260 on the occlusion device 200 is radiopaque.

With reference to FIG. 2, the mesh body 210 of the occlusion device 200 may have an expanded angle ranging from about 30 degrees to about 160 degrees. In some embodiments, the mesh body 210 of the occlusion device 200 may have an expanded angle angle ranging from about 30 degrees to about 135 degrees. In some embodiments, the mesh body 210 of the occlusion device 200 may have an expanded angle ranging from about 50 degrees to about 135 degrees, or ranging from about 90 degrees to about 135 degrees, or ranging from about 125 degrees to about 135 degrees. In various embodiments, the mesh body 210 of the occlusion device 200 may have a width ranging from about 5 mm to about 15 mm when the mesh body is unconstrained.

With reference to FIG. 3, an example occlusion device 300 according to alternative embodiments of the disclosure is now described. The occlusion device 300 shown in FIG. 3 is similar to the occlusion device 200 shown in FIG. 2 in many aspects, except that the ends or tips 318 of the inner braid 316 extend out of the distal pinch member 352 and are terminated with an outward inversion, forming a flower-shaped configuration. The flower-shaped termination may serve as an atraumatic tip of the occlusion device 300, reducing or avoiding the risk of rupturing the aneurysm or other tissue during deployment when the occlusion device 300 is pushed upward.

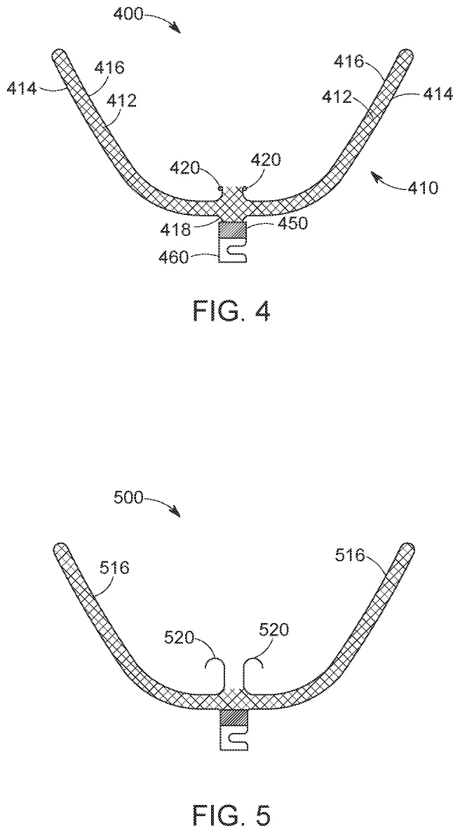

With reference to FIG. 4, an example occlusion device 400 according to alternative embodiments of the disclosure is now described. The occlusion device 400 shown in FIG. 4 comprises a double-layered mesh body 410 including a first or outer braid layer 414 and a second or inner braid layer 416. The double-layered mesh body 410 can be formed by folding plural braided strands in an initial e.g., cylindrical form over themselves along a circumferential line, bringing one ends or second ends 420 of the plural braided strands 412 adjacent to the other ends or first ends 418 of the plural braided strands 412. The mesh body 410 or at least a portion of the mesh body 410, when deployed, has pores with a pore size, that can “divert” a flow of a fluid such as a blood flow, or inhibit the blood flow through the mesh body or at least a portion of the mesh body into an aneurysm or other treatment site to a degree sufficient to lead to thrombosis and healing of the aneurysm or other tissue.

As shown in FIG. 4, the end portions 418 of the first or outer braid 414 can be clamped by a pinch member 450. The pinch member 450 can be invisible to an imaging system commonly used in the art such as non-radiopaque. The end portions 420 of the second or inner braid 416 are un-pinched. The braided strands 412 may include radiopaque strands, such as radiopaque DFT wires. The use of radiopaque wires allows visualization of the occlusion device, especially at the pinch points 450 where the wires are bundled and clamped and the density of the radiopaque materials is greater, thereby eliminating the need for an independent or separate radiopaque marker band. As shown in FIG. 4, the occlusion device 400 may also include a coupler 460 attached to the pinch member 450. The coupler 460 may be constructed or configured to attached to a complementary coupler on a delivery system, and is capable of being detached mechanically or electrolytically or in other suitable means. The coupler 460 on the delivery system can be radiopaque. The coupler 460 on the occlusion device 400 may be non-radiopaque. Alternatively, the coupler 460 on the occlusion device 400 can be radiopaque.

With reference to FIG. 4, the mesh body 410 of the occlusion device 400 may have an expanded angle ranging from about 30 degrees to about 160 degrees. In some embodiments, the mesh body 410 of the occlusion device 400 may have an expanded angle angle ranging from about 30 degrees to about 135 degrees. In some embodiments, the mesh body 410 of the occlusion device 400 may have an expanded angle ranging from about 50 degrees to about 135 degrees, or ranging from about 90 degrees to about 135 degrees, or ranging from about 125 degrees to about 135 degrees. In various embodiments, the mesh body 410 of the occlusion device 400 may have a width ranging from about 5 mm to about 15 mm when the mesh body is unconstrained.

With reference to FIG. 5, an example occlusion device 500 according to alternative embodiments of the disclosure is now described. The occlusion device 500 shown in FIG. 5 is similar to the occlusion device 400 shown in FIG. 4 in many aspects, except that the end portions 520 of the second or inner braid 516 are terminated with an outward inversion, forming a flower-shaped configuration. The flower-shaped termination may serve as an atraumatic tip of the occlusion device 500, avoiding or reducing the risk of rupturing the aneurysm or other tissue during deployment when the occlusion device is pushed upward.

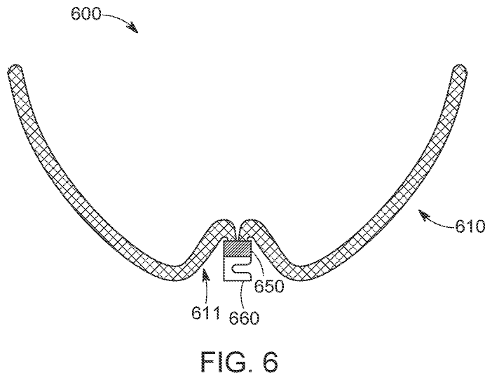

With reference to FIG. 6, an example occlusion device 600 according to alternative embodiments of the disclosure is now described. The occlusion device 600 shown in FIG. 6 is similar to the occlusion device 100 shown in FIG. 1A in many aspects, except that the mesh body 610 comprises a recessed portion 611 at the bottom. The recessed portion 611 allows the pinch member 650 and the coupler 660 or at least a portion of the coupler 660 to be nested in or surrounded by the occlusion device 600 when deployed, or allow at least a portion of the detachment zone to be pulled away from a parent vessel. The coupler 660 attached to the pinch member 650 may therefore remain in the aneurysm rather than protrude into a parent vessel, thereby avoiding or reducing the risk of creating thrombogenic events. The geometry of the recessed portion 611 can be formed during heat-setting of the occlusion device.

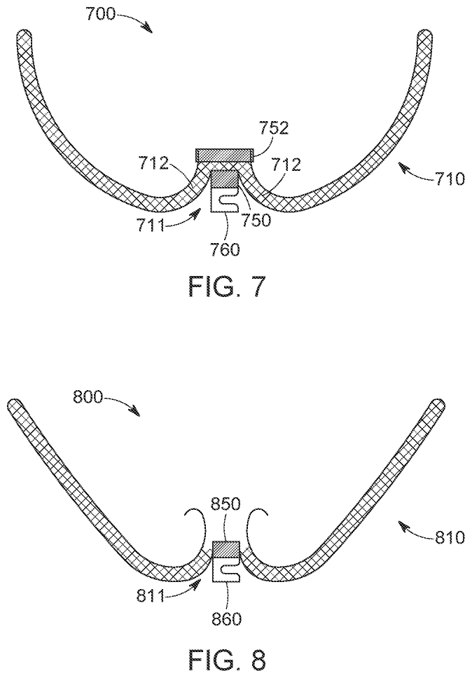

With reference to FIG. 7, an example occlusion device 700 according to alternative embodiments of the disclosure is now described. The occlusion device 700 shown in FIG. 7 is similar to the occlusion device 200 shown in FIG. 2 in many aspects, except that the mesh body 710 comprises a recessed portion 711 at the bottom. The recessed portion 711 allows the proximal pinch member 750 and the coupler 760, or the pinch member 750 and at least a portion of the coupler 760 attached to the proximal pinch member 750 to be nested in the occlusion device 700 when deployed, or allow at least a portion of the detachment zone to be pulled away from a parent vessel. The proximal pinch member 750 or the coupler 760 or at least a portion of the coupler 760 attached to the proximal pinch member 750 may therefore remain in the aneurysm rather than protrude into a parent vessel, thereby avoiding or reducing the risk of creating thrombogenic events. The recessed portion 711 also allows the proximal pinch member 750 to be positioned adjacent to the distal pinch member 752, thereby increasing the density of the braded strands 712 near the detachment zone. The increased density of radiopaque strands 712 near the detachment zone allows better visualization of the occlusion device 700 during delivery and deployment of the device.

With reference to FIG. 8, an example occlusion device 800 according to alternative embodiments of the disclosure is now described. The occlusion device 800 shown in FIG. 8 is similar to the occlusion device 500 shown in FIG. 5 in many aspects, except that the mesh body 810 comprises a recessed portion 811 at the bottom. The recessed portion 811 allows the pinch member 850 and the coupler 860 or at least a portion of the coupler 860 attached to the pinch member 850 to be nested in the occlusion device 800 when deployed. The pinch member 850 and the coupler 860 or at least a portion of the coupler 860 attached to the pinch member 850 may therefore remain in the aneurysm rather than protrude into a parent vessel, or allow at least a portion of the detachment zone to be pulled away from a parent vessel, thereby avoiding or reducing the risk of creating thrombogenic events. The recessed portion 811 also allows the end portions of the outer braid to be positioned adjacent to the end portions of the inner braid, thereby increasing the density of the braded strands near the detachment zone. The increased density of radiopaque strands near the detachment zone allows better visualization of the occlusion device 800 during delivery and deployment.

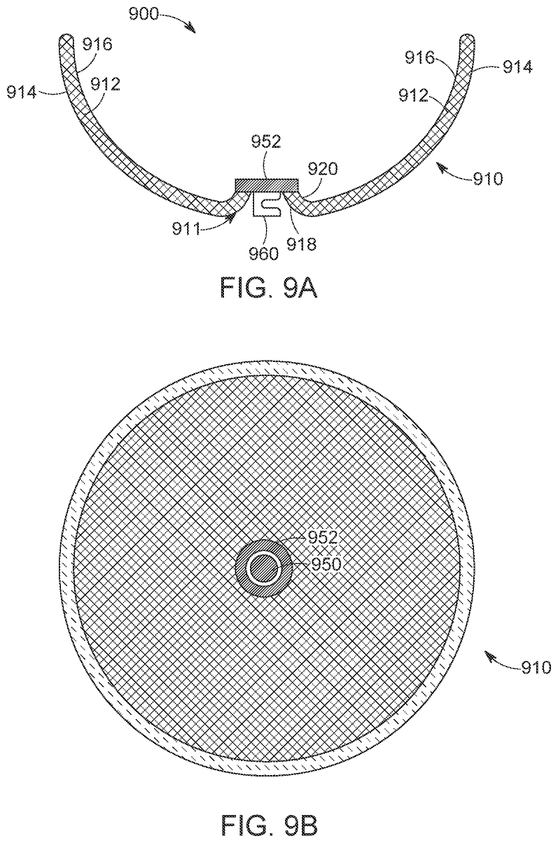

With reference now to FIGS. 9A-9B, an example occlusion device 900 according to alternative embodiments of the disclosure is now described. The occlusion device 900 shown in FIGS. 9A-9B may comprise a double-layer mesh body 910 including a first or outer braid layer 914 and a second or inner braid layer 916. The double-layered mesh body 910 can be formed by folding plural braided strands e.g., in an initial cylindrical form over itself along a circumferential line, bringing one ends of the plural braided strands adjacent to the other ends of the plural braided strands. The mesh body 910 or at least a portion of the mesh body 910, when deployed, has pores with a pore size that can “divert” a flow of a fluid such as a blood flow, or inhibit the blood flow through the mesh body or at least a portion of the mesh body into an aneurysm or other treatment site to a degree sufficient to lead to thrombosis and healing of the aneurysm or other tissue.

As shown in FIGS. 9A-9B, the end portions 918 of the outer braid 914 can be clamped by a first or proximal pinch member 950 (FIG. 9B). The end portions 920 of the inner braid 916 can be clamped by a second or distal pinch member 952. Either or both the first pinch member 950 and the second pinch member 952 may be constructed from a material invisible to an imaging modality such as a non-radiopaque material. The braided strands 912 may include radiopaque strands, such as radiopaque DFT wires. The use of radiopaque wires allows visualization of the occlusion device 900, especially at the pinch points 950, 922 where the wires are bundled and clamped and the density of the radiopaque materials is greater, thereby eliminating the need for an independent or separate radiopaque marker band. The occlusion device 900 may also include a coupler 960 attached to the proximal pinch member 950 for coupling to a delivery system. The coupler 960 may be configured to couple to a complementary coupler on a delivery system, and is capable of being detached mechanically or electrolytically or in other suitable means. The coupler 960 on the delivery system can be radiopaque. The coupler 960 on the occlusion device 900 may be non-radiopaque. Alternatively, the coupler 960 on the occlusion device 950 can be radiopaque.

With reference still to FIGS. 9A-9B, the distal pinch member 952 may comprise an annular band or ring defining a void (FIG. 9B). The annular band 952 may be sized or configured such that the void defined by the annular band 952 can accommodate the proximal pinch member 950 and/or at least a portion of the coupler 960 therein. This design allows the the proximal pinch point 950 and the distal pinch point 952 to be brought closer, further increasing the density of the braded strands 912 near the detachment zone. The increased density of radiopaque strands near the detachment zone allows better visualization of the occlusion device during delivery and deployment.

With reference still to FIGS. 9A-9B, the mesh body 910 comprises a recessed portion 911 at the bottom. The recessed portion 911 allows the proximal pinch member 950 and the coupler 960 or at least a portion of the coupler 960 attached to the pinch member 950 to be nested in the occlusion device 900 when deployed. The proximal pinch member 950 or the coupler 960 attached to the proximal pinch member 950 may therefore remain in the aneurysm rather than protrude into a parent vessel or allow at least a portion of the detachment zone to be pulled away from a parent vessel, thereby avoiding or reducing the risk of creating thrombogenic events.

With reference to FIG. 9A, the mesh body 910 of the occlusion device 900 may have an expanded angle ranging from about 30 degrees to about 160 degrees. In some embodiments, the mesh body 910 of the occlusion device 900 may have an expanded angle angle ranging from about 30 degrees to about 135 degrees. In some embodiments, the mesh body 910 of the occlusion device 900 may have an expanded angle ranging from about 50 degrees to about 135 degrees, or ranging from about 90 degrees to about 135 degrees, or ranging from about 125 degrees to about 135 degrees. In various embodiments, the mesh body 910 of the occlusion device 900 may have a width ranging from about 5 mm to about 15 mm when the mesh body is unconstrained.

With reference to FIG. 10, a method of transferring an occlusion device, e.g., occlusion device 100 into a catheter is now described. The mesh body 110 of the occlusion device 100 in a 3D geometry may be folded into a collapsed configuration for transferring into a catheter. For example, the mesh body 110 in a 3D geometry may be compressed toward a central axis 111 of the occlusion device 100, as indicated by the arrows, into a collapsed configuration. This method can be used for delivery of various embodiments of the occlusion device of the disclosure, including the occlusion devices 100, 200, 300, 400, 500, 600, 700, 800, and 900, no matter whether the distal ends and the proximal ends of the braided strands are secured by a single pinch member or by two separate pinch members.

With reference to FIGS. 11A and 11B, an alternative method of transferring an occlusion device e.g., an occlusion device 300 into a catheter is now described. The mesh body 310 of the occlusion device 300 in a 3D geometry may be stretched into a collapsed configuration for transferring into a catheter. For example, the mesh body 310 in a 3D geometry may be stretched between the proximal pinch point 350 and the distal pinch point 352 in opposite directions, as indicated by the arrows, to form a collapsed configuration. This method can be used for occlusion devices, e.g., those shown in FIGS. 2-3 etc., where the distal ends of the braided strands and the proximal ends of the braided strands are not secured by a single pinch member.

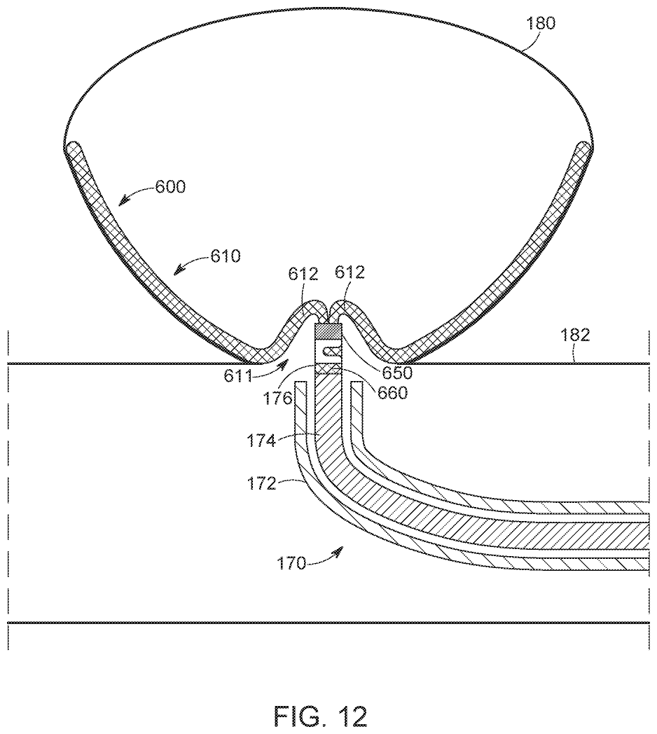

With reference to FIG. 12, an example method for deploying an example occlusion device 600 according to embodiments of the disclosure is now described. As shown, a delivery system 170 may be used to deliver and deploy the occlusion device 600 at a target site for treating e.g., an aneurysm 180 formed on a wall of a vessel 182 e.g., in the neuro vasculature of a patient. Various delivery systems are known and readily available in the art, and therefore their detailed description is omitted herein to focus on description of embodiments of the disclosure. In general, a delivery system 170 includes a catheter or microcatheter 172 for carrying an occlusion device 600 in a collapsed or delivery configuration through the vasculature to a treatment site, a wire element 174 for pushing and/or retracting the occlusion device 600, and a coupler 176 for coupling a complementary coupler 660 on the occlusion device 600. The coupling between the delivery system 170 and the occlusion device 600 may be decoupled via various mechanisms known in the art, including but not limited to mechanical, electrolytical, and hydraulic mechanism, or the like.

For delivery, the occlusion device 600 may be folded into a collapsed configuration, as illustrated in FIGS. 10 and 11A-11B, and constrained within the catheter 172 of the delivery system 170. Once the delivery system 170 is guided and reaches to the neck of the aneurysm 180, the occlusion device 600 can be pushed by a wire element 174 and exit the catheter 172 into the aneurysm 180. Upon release from the catheter 172, the occlusion device 600 may assume a 3D configuration in the aneurysm 180. The occlusion device 600 may be detached from the delivery system 170 by applying electrolytic power or by activating a mechanical detachment mechanism, or other suitable means. For illustration, the occlusion device 600 includes a coupler 660 attached to a pinch member 650 on the occlusion device 600 and connected to a complementary coupler 176 on the delivery system 170. The coupler 176 on the delivery system 170 can be radiopaque or include a marker to facilitate visualization of the occlusion device 600 during the delivery and deployment by imaging the detachment zone. The coupler 660 on the occlusion device 600 may be invisible to an imaging system e.g., non-radiopaque and remain in the aneurysm 180 after the occlusion device 600 is decoupled from the delivery system 170 and deployed in the aneurysm 180. Because the braided strands 612 or at least some of the strands are radiopaque, the occlusion device 600 can still be visualized via e.g., x-ray fluoroscopy, especially at the pinch point 650 due to the increased density of the strands clamped at the end portions. Alternatively, the coupler 660 on the occlusion device 600 can be constructed from a radiopaque material. As shown, the mesh body 610 of the expanded occlusion device 600 may have a recessed portion 611 at the bottom to allow the coupler 660 or at least a portion of the coupler 660 to be nested in the occlusion device 600 within the aneurysm 180, or pulled away from the parent vessel 182 and as such, the risk of thrombogenic events, which might be caused by protrusion of the coupler 650 into the parent vessel, may be reduced or avoided. After deployment, the delivery system 170 and the coupler 176 on the delivery system 170 may be withdrawn from the vasculature. The occlusion device 600 assumes its 3D geometry. The expanded occlusion device 600 in 3D geometry may generally conform to the shape of the aneurysm 180, or a portion of the aneurysm 180 adjacent to the neck, to allow the mesh body 610 to be in contact with the internal wall and cover or bridge the neck of the aneurysm. As such, a single occlusion device can pack and/or cover the aneurysm, inhibiting a blood flow into the aneurysm and promote clotting and subsequent healing.

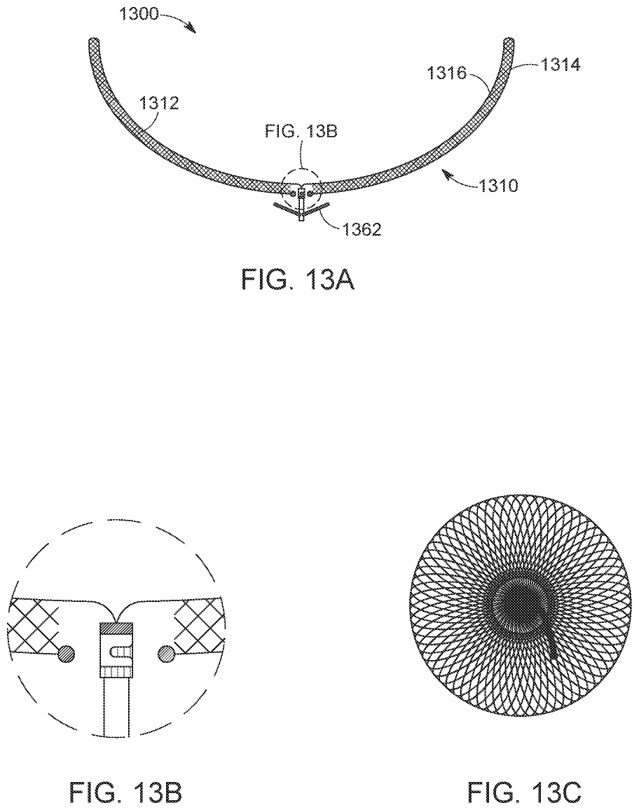

With reference now to FIGS. 13A-13C, an example occlusion device 1300 according to embodiments of the disclosure is now described. In general, the occlusion device 1300 comprises a mesh body 1310 and a pinch member 1350. The mesh body 1310 may be constructed from plural strands 1312. The plural strands 1312 may be braided to form the mesh body 1310. The mesh body 1310 may be a double-layered braided mesh body, including a first or outer braid or layer 1314 and a second or inner braid or layer 1316 as shown. The double-layered mesh body 1310 can be formed by folding the plural strands 1312 e.g., initially in a cylindrical form outwardly over themselves to bring one ends of the plural strands adjacent to the other ends of the plural strands, or to bring the end portions 1318 of the outer braid layer 1314 adjacent to the end portions 1320 of the inner braid layer 1316. The end portions 1320 of the inner braid 1316 may be clamped by the pinch member 1350, whereas the end portions 1318 of the outer braid 1318 are un-pinched, defining an opening or hole 1319 around the pinch member 1350. In some embodiments, the end portions 1318 of the outer braid 1314 may be attached to a ring structure 1321, as better viewed in FIGS. 13B-13C.

With reference to FIG. 13A, the mesh body 1310 of the occlusion device 1300 may have an expanded angle ranging from about 30 degrees to about 160 degrees. In some embodiments, the mesh body 1310 of the occlusion device 1300 may have an expanded angle angle ranging from about 30 degrees to about 135 degrees. In some embodiments, the mesh body 1310 of the occlusion device 1300 may have an expanded angle ranging from about 50 degrees to about 135 degrees, or ranging from about 90 degrees to about 135 degrees, or ranging from about 125 degrees to about 135 degrees. In various embodiments, the mesh body 1310 of the occlusion device 1300 may have a width ranging from about 5 mm to about 15 mm when the mesh body is unconstrained.

Still with reference to FIGS. 13A-13C, similar to some of the other embodiments, the plural strands 1312 may comprise wires constructed of a radiopaque material. For example, the plural strands 1312 for constructing the occlusion device 1300 may comprise drawn filled tubing (DFT) wires, which include a core metal of a radiopaque material such as platinum, gold, tantalum, tungsten, etc., and an outer sheath of a shape-memory material such as Nitinol or other elastic metal alloys. The use of radiopaque DFT wires for the braided strands can reduce or eliminate the need for a radiopaque marker band on an occlusion device. The pinch member 1350 clamping the end portions 1320 of the inner braid or layer 1316 may be constructed from a non-radiopaque material.

Still with reference now to FIGS. 13A-13C, the occlusion device 1300 may include a coupler 1360 attached to the pinch member 1350 for coupling the occlusion device 1300 to a delivery system. The coupler 1360 attached to the pinch member 1350 can also be constructed from a non-radiopaque material. The coupler 1360 on the occlusion device 1300 may couple with a complementary coupler on a delivery system. The complementary coupler on the delivery system may be radiopaque to facilitate visualization of the occlusion device 1300 during delivery and deployment. The coupler 1360 on the occlusion device 1300 and the coupler on the delivery system may be constructed or configured such that they can be detached from each other upon application of electrolytic power or by activating a mechanical detachment mechanism. According to embodiments of the disclosure, the delivery system may include a flap 1362 (FIG. 13A) at its distal end to facilitate folding or retracting of the occlusion device 1300 into the delivery system. The flap 1362 may be constructed from a flexible and/or lubricious material such as PTFE, ePTFE, or any other suitable polymeric materials.

Advantageously, the occlusion device 1300 of the disclosure allows the pinch member 1350 and the detachment zone to be inherently receded or nested within the hole or opening 1319 defined or created by the un-pinched end portions 1318 of the outer braid 1314. As such, the pinch member 1350 and the coupler 1360 or at least a portion of the coupler 1360 can remain with the occlusion device 1300 in the aneurysm to be treated rather than protrude into a parent vessel, thereby reducing or avoiding the risk of causing thrombogenic events.

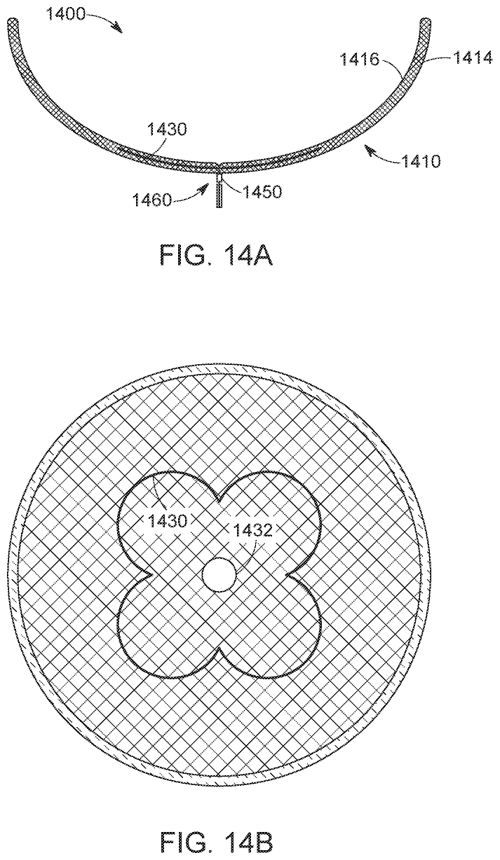

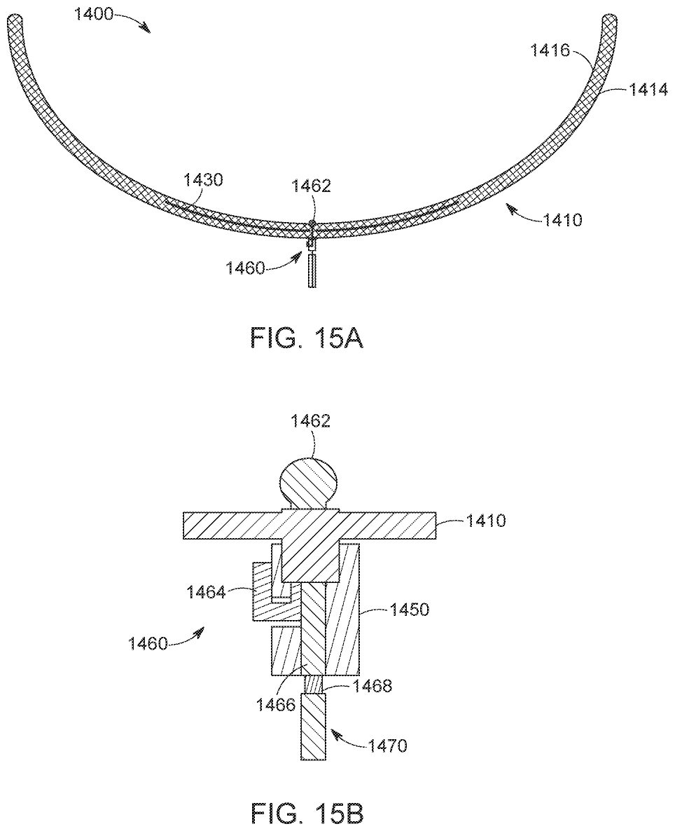

With reference to FIGS. 14A-14B and 15A-15B, an example occlusion device 1400 according to embodiments of the disclosure is now described. The occlusion device 1400 shown in FIGS. 14A-14B and 15A-15B is similar to the occlusion device 100 shown in FIGS. 1A-1B in many aspects, except that the occlusion device 1400 shown in FIGS. 14A-14B and 15A-15B includes a flexible filler layer 1430 between the first or outer braid layer 1414 and the second or the inner braid layer 1416 and a unique coupling system 1460.

With reference to FIGS. 14A-14B, the filler layer 1430 may be constructed or configured to facilitate folding of the braided mesh body 1410 when retracted into a catheter or sheath. The filler layer 1430 may have a geometry in the form of a clover, a three-petal, a disk with cuts, or other suitable forms which can be readily folded and retracted into a catheter or sheath. The filler layer 1430 may be provided with a hole 1432 at its geometrical center and aligned with the pinch member 1450 or pinch point of the braids so that the filler layer 1430 can remain centered. The filler layer 1430 can be constructed or configured to increase coverage of the aneurysm neck or divert a fluid flow away from the treatment site such as an aneurysm. The filler layer 1430 may be constructed from a flexible polymer such as PTFE, ePTFE, or other suitable polymers. The flexible filler layer 1430 may also be constructed of a suitable metallic foil. The filler layer 1430 can be sized and shaped so that it does not fully cover the neck of the aneurysm to avoid pressurizing the aneurysm. Alternatively, the filler layer 1430 can be sized and shaped to fully cover the neck of the aneurysm. For example, the filler layer 1430 can be sized to be larger than the neck of an aneurysm to isolate the aneurysm from the parent vessel to cause immediate clotting. The filler layer can be in the form of a disk without cuts. This can be useful in treating ruptured aneurysms to provide immediate protection against re-rupture. One of ordinary skill in the art will appreciate that while embodiments of a filler layer are described herein in connection with FIGS. 14A-14B and 15A-15B, the occlusion devices shown in FIGS. 1A through 13C and 15A through 16B can also include or be modified to include a filler layer as described herein.

With reference to FIG. 14A, the mesh body 1410 of the occlusion device 1400 may have an expanded angle ranging from about 30 degrees to about 160 degrees. In some embodiments, the mesh body 1410 of the occlusion device 1400 may have an expanded angle angle ranging from about 30 degrees to about 135 degrees. In some embodiments, the mesh body 1410 of the occlusion device 1400 may have an expanded angle ranging from about 50 degrees to about 135 degrees, or ranging from about 90 degrees to about 135 degrees, or ranging from about 125 degrees to about 135 degrees. In various embodiments, the mesh body 1410 of the occlusion device 1400 may have a width ranging from about 5 mm to about 15 mm when the mesh body is unconstrained.

With reference to FIGS. 15A-15B, according to embodiments of the disclosure, the occlusion device 1400 may comprise a coupler 1460 configured to couple the occlusion device 1400 with a delivery system 1470. As shown, the coupler 1460 may include a spherical body 1462 e.g., in a ball shape and two wires 1464, 1466 each having a distal end being attached to the spherical body 1462. The spherical body 1462 can be located at the clamped end portions or pinch point of the inner braid 1416 and provided with an interference fit with the clamped end portions. The first wire 1464 may be attached to the pinch member 1450 e.g., by threading through a slot at a side of the pinch member 1450. The proximal end of the first wire 1464 may be further affixed to the pinch member 1450 by adhesive, soldering, or other suitable means. The second wire 1466 may extend through the pinch member 1450 and coupled to a delivery system 1470. The proximal end of the second wire 1466 and a distal end of the delivery system 1470 may form an ablation zone 1468 capable of being disintegrated by applying electrolytically power. Alternatively, the proximal end of the second wire 1466 may have a mechanical feature such as a hook, slot, thread or the like, configured to couple with a complementary mechanical feature on the distal end of the delivery system 1470, the coupling of which can be decoupled by actuating a mechanical mechanism.

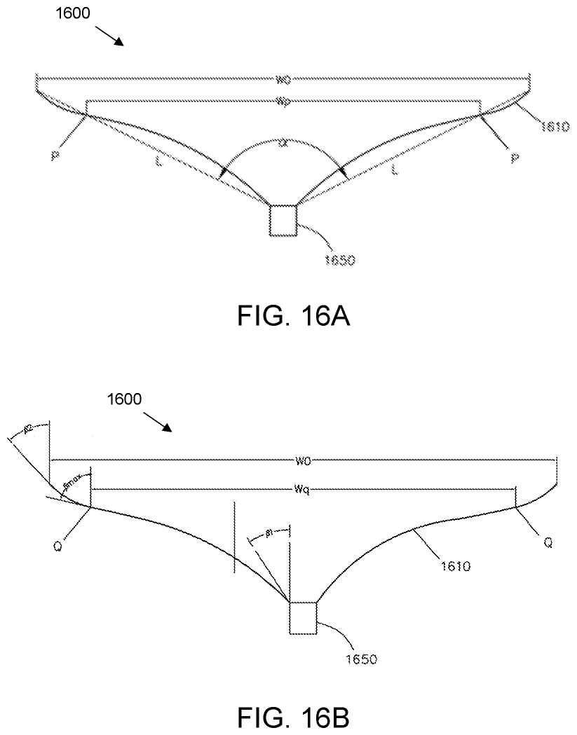

As shown in FIG. 16A and FIG. 6B, an example occlusion device 1600 in an expanded configuration according to alternative embodiments of the disclosure is now described. The occlusion device 1600 as shown in FIG. 16A and FIG. 16B differs from the embodiments described above in respect of the shape of a braided mesh body 1610 of the occlusion device 1600. The shape of contour lines (outer contour lines) of the braided mesh body as shown in FIG. 16A and FIG. 16B will be illustrated below. It should be explained that in a contour line of the braided mesh body 1610 as shown in FIG. 16A and FIG. 16B, an end portion (a base end) on a side away from a pinch member 1650 is located at the highest point of the entire braided mesh body 1610, and an end portion (a tail end) on a side closer to the pinch member 1650 is located at an adjacent point of the braided mesh body 1610 just adjacent to the pinch member 1650.

Specifically, as shown in FIG. 16A, the contour line of the braided mesh body 1610 of the occlusion device 1600 according to the embodiments of the disclosure is formed in a curve shape extending from the base end to the tail end, and as shown in FIG. 16B, the inclination of the braided mesh body 1610 gradually increases and then gradually decreases as the braided mesh body 1610 extends from the base end to the tail end, in the expanded configuration, wherein the inclination is defined as the degree of inclination of a tangential direction of any point on the braided mesh body 1610 with respect to a vertical direction, and the magnitude of the included angle between the tangential direction and the vertical direction can represents the magnitude of the inclination.

The braided mesh body 1610 is configured in such a way that (the description of the shape of the “contour line” below typically involving a contour line on a single side): a ratio Lc/La of the length of a line L connecting two ends (the base end and the tail end) of the contour line (that is, a straight-line distance between two ends of the contour line, hereinafter referred to as “the chord length Lc of the contour line”) to the length of the contour line itself (hereinafter referred to as “the arc length La of the contour line”) is greater than or equal to 0.8, that is, not less than 0.8, for example, the ratio may be within the following ranges: from 0.8 to 0.99, from 0.8 to 0.95, etc. For ease of illustration, the ratio of the chord length Lc of the contour line to the arc length La of the contour line is defined as “a chord-to-arc ratio Lc/La” hereinafter.

Compared with a situation where a chord-to-arc ratio is smaller (a situation where difference in lengths of a chord and an arc is greater due to a greater deviation distance therebetween), by means of setting the value of the chord-to-arc ratio Lc/La as stated above, the spacing between the chord and the arc is smaller in the embodiments of the disclosure, such that the shape of the overall braided mesh body 1610 tends to be flat, making the braided mesh body 1610 closely abuts against the inner wall of the aneurysm more easily and bridged at the neck of the aneurysm more stably. Moreover, it prevents the braided mesh body 1610 from displacing from the neck of the aneurysm, thus the package and/or coverage of the aneurysm is facilitated, inhibiting a blood flow into the aneurysm and promoting clotting and subsequent healing.

In an embodiment of the disclosure, the chord-to-arc ratio Lc/La may preferably be greater than or equal to about 0.9. For example, the chord-to-arc ratio Lc/L may be from about 0.90 to about 0.99. The chord-to-arc ratio Lc/La may more preferably be from about 0.95 to about 0.99. For example, the chord-to-arc ratio Lc/La may be about 0.91, 0.92, 0.93, 0.94, 0.95, 0.96 or 0.97.

In the embodiments of the disclosure, as shown in FIG. 16A, since the contour line of the braided mesh body 1610 of the occlusion device 1600 of the embodiments of the disclosure is formed in such a way that the inclination gradually increases and then gradually decreases from the base end to the tail end as stated above, there is an intersection point P (also referred as “a first position”) between the contour line of the braided mesh body 1610 and a straight line L connecting the base end and the tail end of the contour line.

As shown in FIG. 16A, the ratio Wₚ/W₀ of the distance or width Wₚ between the intersection points P on left and right contour lines (that is, “a width of the points P (the first position)”) to the maximal width W₀ of the overall braided mesh body 1610 of the occlusion device 1600 is greater than or equal to 0.5. For example, the ratio Wₚ/W₀ may be within a range from 0.5 to 0.95, or within a range from 0.5 to 0.9, for example.

In an embodiment of the disclosure, by means of setting the range of Wₚ/W₀ as stated above, the point P is provided at a position closer to an outer side in a left-right direction compared with the situation where the Wₚ/W₀ is smaller. Therefore, the overall mesh body may tend to have a flat shape, in particular, parts of the mesh body that are located outer sides in the left-right direction closely abut against the inner wall of the aneurysm more easily, and the mesh body may be bridged at the neck of the aneurysm more stably. Moreover, the overall mesh body can be prevented from displacing from the neck of the aneurysm. Therefore, the package and/or coverage of the aneurysm is facilitated, inhibiting a blood flow into the aneurysm and promoting clotting and subsequent healing.

In an embodiment of the disclosure, the range of Wₚ/W₀ may preferably be from about 0.8 to 0.9, and the range of Wₚ/W₀ may more preferably be from about 0.85 to 0.9. In addition, the value of Wₚ/W₀ may also be about 0.4 to about 0.6, and may even be, for example, 0.1 to 0.6. The design can be made according to the specific application situation.

In addition, as shown in FIG. 16B, since the inclination of the braided mesh body 1610 gradually increases and then gradually decreases from the base end to the tail end, a maximal-inclination point Q (also referred as “a second place”) exists in the braided mesh body 1610 in the contour line of the braided mesh body 1610 in the expanded configuration. Herein, the inclination at the base end of the braided mesh body 1610 in the expanded configuration is defined as a base end inclination β₁, the inclination at the point Q is defined as a maximal inclination βₘₐₓ, and the inclination at the tail end is defined as a tail end inclination β₂.

In the embodiments of the disclosure, as shown in FIG. 16B, the braided mesh body 1610 is configured in such a way that the inclination thereof in the expanded configuration gradually increases from the base end to the point Q. The shape near the base end of the braided mesh body 1610 that is configured in this way is fitted to the inner wall around the neck of the aneurysm, compared with the situation where the inclination keeps unchanged or gradually decreases, so as to facilitate the bridging of the braided mesh body at the neck of the aneurysm, further packing and/or covering the aneurysm, inhibiting a blood flow into the aneurysm and promoting clotting and subsequent healing.

In another aspect, as shown in FIG. 16B, the braided mesh body 1610 is configured in such a way that the inclination thereof gradually decreases from the point Q to the tail end, such that the braided mesh body 1610 is contracted to inner sides in the left-right direction near the tail end, so as to avoid interference between the tail end of the braided mesh body 1610 and, for example, the inner wall of the aneurysm, and avoid the wear or breakage of the device and the inner wall of the aneurysm due to the interference, improving the compliance of the braided mesh body 1610.

In the embodiments of the disclosure, as shown in FIG. 16B, the range of the base end inclination β₁ is from about 10 degrees to about 40 degrees, the range of the tail end inclination β₂ is from about 15 degrees to about 70 degrees, and the range of the maximal inclination βₘₐₓ is from about 70 degrees to about 100 degrees.

Compared with the situation where the base end inclination β₁ is formed greater, in the embodiments of the disclosure, it facilitates the constraint (contraction) of the braided mesh body 1610 in a catheter 172 of a delivery system 170 when in use (referring to FIG. 10, FIG. 11A and FIG. 11B), by means of configuring the braided mesh body 1610 as stated above such that the range of the base end inclination β₁ thereof in the expanded configuration is formed ranging from about 10 degrees to about 40 degrees.

Compared with the situation where the maximal inclination βₘₐₓ is smaller, the overall mesh body may tend to have a flat shape to closely abut against, for example, the inner wall of the aneurysm more easily, and be bridged at the neck of the aneurysm, by means of configuring the braided mesh body 1610 as stated above such that the range of the maximal inclination βₘₐₓ thereof in the expanded configuration is from about 70 degrees to about 100 degrees, so as to facilitate the package and/or coverage of the aneurysm, and to inhibit a blood flow into the aneurysm and to promote clotting and subsequent healing.

By means of configuring the braided mesh body 1610 as stated above such that the range of the tail end inclination β₂ thereof in the expanded configuration is from about 15 degrees to about 70 degrees, the tail end of the braided mesh body 1610 closely abuts against, for example, the inner wall of the aneurysm more easily, and the interference between the tail end and the inner wall of the aneurysm is avoided, thus avoiding the damage to the braided mesh body 1610 and the inner wall of the aneurysm.

In an embodiment of the disclosure, the range of the base end inclination β₁ is preferably from about 10 degrees to about 30 degrees. The range of the base end inclination β₁ is more preferably from about 15 degrees to about 30 degrees.

In an embodiment of the disclosure, the range of the maximal inclination βₘₐₓ is preferably from about 85 degrees to about 95 degrees, or from about 80 degrees to about 90 degrees. The range of the maximal inclination βₘₐₓ is more preferably from about 85 degrees to about 90 degrees, or from about 90 degrees to about 95 degrees.

In an embodiment of the disclosure, the range of the tail end inclination β₂ is preferably from about 40 degrees to about 70 degrees. The range of the tail end inclination β₂ is more preferably from about 40 degrees to about 60 degrees.

In addition, as shown in FIG. 16B, the ratio Wₚ/W₀ of the distance Wₚ (that is, “a width of the point Q (the second position)”) between the maximal-inclination points Q on left and right contour lines to the maximal width W₀ of the overall braided mesh body 1610 of the occlusion device is greater than or equal to 0.5. For example, the Wₚ/W₀ may be within the range from 0.5 to 0.9, or within the range from 0.5 to 0.98.

Compared with the situation where the value of Wₚ/W₀ is smaller, in the embodiment of the disclosure, by means of configuring the braided mesh body 1610 as stated above, the maximal-inclination points Q of the braided mesh body 1610 are closer to the outer sides in the left-right direction, that is, the maximal-inclination points Q which is the inclination conversing point is closer to the outer sides in the left-right direction. Therefore, the overall mesh body may tend to have a flat shape, in particular, parts of the mesh body that are closer to the outer sides in the left-right direction closely abut against the inner wall of the aneurysm more easily, and the mesh body may be bridged at the neck of the aneurysm more stably. Moreover, the overall mesh body can be prevented from displacing from the neck of the aneurysm. Therefore, the package and/or coverage of the aneurysm is facilitated, inhibiting a blood flow into the aneurysm and promoting clotting and subsequent healing.

In an embodiment of the disclosure, the range of Wₚ/W₀ may preferably be from about 0.7 to 0.95, and the range of Wₚ/W₀ may more preferably be from about 0.8 to 0.95.

According to the embodiments described above, the range of the maximal width W₀ of the braided mesh body 1610 is from about 5 mm to 15 mm when the braided mesh body 1610 is unconstrained in the expanded configuration. As examples, the mesh body 110 of the occlusion device 100 may have a width of 5 mm, 7 mm, 9 mm, 11 mm, 14 mm and 15 mm, or any width therebetween. Alternatively, the mesh body 110 of the occlusion 100 may have a width smaller than 5 mm and/or greater than 15 mm.

According to the embodiments described above, an included angle between a connecting line L of two ends (the base end and the tail end) of a left contour line and a connecting line L of two ends (the base end and the tail end) of a right contour line is defined as a contour lines included angle α between the left contour line and the right contour line of the braided mesh body 1610 (refer to FIG. 16A), the range of which is from about 90 degrees to about 170 degrees in the embodiments of disclosure. In some embodiments, the braided mesh body 1610 of the occlusion device 1600 may have a contour line included angle α ranging from about 100 degrees to about 170 degrees, or ranging from about 120 degrees to about 170 degrees, or ranging from about 120 degrees to about 160 degrees. In various embodiments of the disclosure, the mesh body 1610 of the occlusion device 1600 may has a contour line included angle of 90 degrees, 100 degrees, 110 degrees, 120 degrees, 130 degrees, 140 degrees, 150 degrees, 160 degrees and 170 degrees, or of any angle therebetween. It should be noted that the above example contour line included angles are provided merely for the purpose of thorough understanding of the disclosure. The scope of the appended claims is not limited to specific angles. Alternatively, the mesh body 1610 of the occlusion device 1600 may have a contour line included angle smaller than 90 degrees, such as a contour line included angle of 60 degrees or 70 degrees, and/or a contour line included angle greater than 170 degrees, such as a contour line included angle of 172 degrees or 175 degrees.

Compared with the situation where the contour line included angle is smaller, in the embodiments of the disclosure, the braided mesh body 1610 may tend to be flat, so as to closely abut against the inner wall of the aneurysm more easily, and the mesh body may be bridged at the neck of the aneurysm more stably, by means of configuring the braided mesh body 1610 as stated above. Moreover, the overall mesh body can be prevented from displacing from the neck of the aneurysm. Therefore, the package and/or coverage of the aneurysm is facilitated, inhibiting a blood flow into the aneurysm and promoting clotting and subsequent healing.

Coatings on Occlusion Devices

According to embodiments of the disclosure, the occlusion device can comprise various materials to enhance the performance of the occlusion device. Polymeric and/or monomeric materials and bioactive agents can be coated on the occlusion device to provide desirable properties, including reduced thrombogenicity, lubricity, drug delivery, and so on.

According to one embodiment of the disclosure, a layer of anti-thrombogenic material may be coated on the braided mesh body or at least a portion of the braided mesh body of the occlusion device to reduce thromboembolic events. Suitable anti-thrombogenic materials include but are not limited to naturally occurring glycosaminoglycan (Heparin), phosphorylcholines (PC) such as methacryloyloxyethyl phosphorylcholine, acryloyloxyethyl phosphorylcholine, and phosphorylcholines-based monomers. Heparin materials are commercially available e.g., from Pfizer, Inc. of New York, NY. Phosphorylcholines materials are commercially available e.g., from NOF Corporation of Tokyo, Japan. An anti-thrombogenic material can be applied to the occlusion device by various methods, including e.g., spraying, dipping, and combination thereof. The preparation of a solution of an anti-thrombogenic material and the process of application of the solution on an occlusion device are generally known and thus their detailed description is omitted herein to focus on description of various embodiments of the disclosure. Based on applications, the thickness of the layer of an anti-thrombogenic material on the occlusion device can range from 1 nanometer to 2000 nanometers.

According to one embodiment of the disclosure, a layer of lubricious material may be coated on the braided mesh body or at least a portion of the braided mesh body of the occlusion device to increase lubricity during delivery and reduce the risks of damage to the devices. Suitable lubricious materials include but are not limited to polytetrafluoroethylene (PTFE), fluorinated ethylene propylene (FEP), and other suitable polymeric materials. PTFE and FEP materials are commercially available e.g., from E. I. du Pont de Nemours and Company of Wilmington, DE. A lubricious material can be applied to the occlusion device by various methods, including e.g., spraying, dipping, and combination thereof. The preparation of a solution of a lubricious material and the process of application of the solution on an occlusion device are generally known and thus their detailed description is omitted herein to focus on description of various embodiments of the disclosure. Based on applications, the thickness of the layer of a lubricious material on the occlusion device can range from 1 nanometer to 2000 nanometers.

Various embodiments of an occlusion device, system, and method have been described with reference to figures. It should be noted that an aspect described in conjunction with a particular embodiment is not necessarily limited to that embodiment and can be practiced in any other embodiments. The figures are intended for illustration of embodiments but not for exhaustive description or limitation on the scope of the disclosure. Alternative structures, components, and materials will be readily recognized as being viable without departing from the principle of the claimed invention.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “proximal” and its grammatically equivalent refers to a position, direction or orientation towards the user or physician's side. The term “distal” and its grammatically equivalent refers to a position, direction or orientation away from the user or physician's side. The term “first” or “second” etc. may be used to distinguish one element from another in describing various similar elements. It should be noted the terms “first” and “second” as used herein include references to two or more than two. Further, the use of the term “first” or “second” should not be construed as in any particular order unless the context clearly dictates otherwise. The term “about” may be used to indicate a value that can include a variation of ±15% of the value modified by the term. The recitation of numerical ranges by endpoints includes all numbers within that range (e.g., 5-15 includes 5, 5.5, 7, 9, 11, 14, 14.5, and 15).

Those skilled in the art will appreciate that various other modifications may be made. All these or other variations and modifications are contemplated by the inventors and within the scope of the invention.

What is claimed is:

1. An occlusion device, comprising:
a braided mesh body comprising plural strands, each strand having a first end portion and a second end portion, the plural strands folding over to bring the second end portions of the plural strands adjacent to the first end portions of the plural strands, forming a double-layer of the braided mesh body; and
at least one pinch member clamping the first end portions and the second end portions of the plural strands, wherein
at least one of the plural strands is radiopaque; and
the at least one pinch member is non-radiopaque.

2. The occlusion device of claim 1, wherein the braided mesh body has an expanded configuration in a bowl shape with a recessed portion at bottom, and wherein the at least one pinch member rests in the recessed portion.

3. The occlusion device of claim 2, further comprising a coupler attached to the at least one pinch member and configured to couple to a delivery device, the coupler being non-radiopaque and capable of being decoupled from the delivery device mechanically or electrolytically.

4. The occlusion device of claim 3, wherein at least a portion of the coupler rests in the recessed portion.

5. The occlusion device of claim 1, wherein the at least one pinch member comprises:
a first pinch member clamping the first end portions of the plural strands; and
a second pinch member clamping the second end portions of the plural strands.

6. The occlusion device of claim 5, wherein the braided mesh body has an expanded configuration in a bowl shape with a recessed portion at bottom, and wherein the first pinch member rests in the recessed portion.

7. The occlusion device of claim 6, further comprising a coupler attached to the first pinch member and configured to couple to a delivery device, the coupler being non-radiopaque and capable of being decoupled from the delivery device mechanically or electrolytically.

8. The occlusion device of claim 7, wherein at least a portion of the coupler rests in the recessed portion.

9. The occlusion device of claim 5, wherein the second pinch member comprises an annular ring defining a void, and wherein at least a portion of the first pinch member rests within the void.

10. The occlusion device of claim 5, wherein tips of the second end portions of the plural strands extend out of the second pinch member and are terminated with an outward inversion, forming a flowered-tip geometry of the braided mesh body.

11. The occlusion device of claim 1, wherein the plural strands are coated with an anti-thrombogenic material.

12. The occlusion device of claim 1, wherein the plural strands are coated with a material comprising glycosaminoglycan (Heparin) or phosphorylcholines (PC).

13. An occlusion device, comprising:
a braided mesh body comprising plural strands, each strand having a first end portion and a second end portion; and
a pinch member clamping first end portions of the plural strands, wherein
the plural strands fold inwardly to bring second end portions of the plural strands adjacent to the first end portions, forming a double-layer of the braided mesh body, and
the second end portions of the plural strands are un-pinched.

14. The occlusion device of claim 13, wherein at least one of the plural strands is radiopaque.

15. The occlusion device of claim 13, wherein the pinch member is non-radiopaque.

16. The occlusion device of claim 13, wherein tips of the second end portions of the plural strands are terminated with an outward inversion, forming a flowered-tip geometry of the braided mesh body.

17. The occlusion device of claim 16, wherein the flowered-tip geometry defines a void, and at least a portion of the pinch member rests within the void.

18. The occlusion device of claim 17, further comprising a coupler attached to the pinch member, wherein the coupler is configured to couple to and capable of being decoupled from a delivery device, and at least a portion of the coupler rests within the void.

19. The occlusion device of claim 13, further comprising a coupler attached to the pinch member, wherein the coupler is configured to couple to and capable of being decoupled from a delivery device, and is non-radiopaque.

20. The occlusion device of claim 13, wherein the plural strands are coated with an anti-thrombogenic material.

21. The occlusion device of claim 13, wherein the plural strands are coated with a material comprising glycosaminoglycan (Heparin) or phosphorylcholines (PC).

22. The occlusion device of claim 13, wherein the braided mesh body has an expanded configuration generally in a bowl shape.

23. The occlusion device of claim 13, wherein
at least one of the plural strands is radiopaque;
the pinch member is non-radiopaque;
tips of the second end portions of the plural strands are terminated with an outward inversion, forming a flowered-tip geometry of the braided mesh body,
the braided mesh body has an expanded configuration generally in a bowl shape with a recessed portion at bottom and the un-pinched second end portions of the plural strands define a void, and
at least a portion of the pinch member rests within the void.

24. An occlusion device, comprising:
a braided mesh body comprising plural strands, each strand having a first end portion and a second end portion; and
a pinch member clamping first end portions of the plural strands, wherein
the plural strands fold outwardly to bring second end portions of the plural strands adjacent to the first end portions, forming a double-layer of the braided mesh body,
the second end portions are un-pinched, defining an opening around the pinch member and allowing at least a portion of the pinch member to rest within a void defined at least by the opening.

25. The occlusion device of claim 24, further comprising a ring structure, wherein the second end portions of the plural strands are attached to the ring structure.

26. The occlusion device of claim 24, wherein the pinch member is non-radiopaque.

27. The occlusion device of claim 24, wherein at least one of the plural strands is radiopaque.

28. The occlusion device of claim 24, further comprising a coupler attached to the pinch member and configured to be coupled to and decoupled from a delivery device.

29. The occlusion device of claim 28, wherein the coupler is non-radiopaque.

30. The occlusion device of claim 29, wherein at least a portion of the coupler rests within the void.

31. An occlusion device, comprising:
a braided mesh body comprising plural strands, each strand having a first end portion and a second end portion, the plural strands folding over to bring second end portions of the plural strands adjacent to the first end portions, forming a double-layer of the braided mesh body;
a pinch member clamping the first end portions and the second end portions of the plural strands; and
a flexible filler layer between the double-layer of the braided mesh body.

32. The occlusion device of claim 31, wherein the flexible filler layer has a hole at a geometrical center of the flexible filler layer to allow the flexible filler layer to be centered at the pinch member.

33. The occlusion device of claim 32, wherein the flexible filler layer is constructed from a polymeric material or a metallic foil.

34. The occlusion device of claim 33, wherein the flexible filler layer comprises two or more sections of a same or similar shape to facilitate folding of the flexible filler layer.

35. The occlusion device of claim 33, wherein the flexible filler layer comprises a deployed configuration having a maximal dimension smaller than a dimension of a neck of an aneurysm to be treated.

36. The occlusion device of claim 33, wherein the flexible filler layer comprises a deployed configuration having a maximal dimension larger than a dimension of a neck of an aneurysm to be treated.

37. The occlusion device of claim 31, wherein the pinch member is non-radiopaque.

38. The occlusion device of claim 37, wherein at least one of the plural strands is radiopaque.

39. The occlusion device of claim 31, further comprising a coupler configured to couple the braided mesh body to a delivery device or decouple the braided mesh body from a delivery device.

40. The occlusion device of claim 39, wherein the coupler comprises a spherical body having an interference fit with the clamped second end portions of the braided mesh body, a first wire having a distal end attached to the spherical body and a proximal end attached to the pinch member, and a second wire having a distal end attached to the spherical body and a proximal end configured to couple the braided mesh body to a delivery device.

41. The occlusion device of claim 40, wherein the pinch member has a slot on a side to allow the proximal end of the first wire to be pull through and fixed to the pinch member.

42. An occlusion device, comprising:
a braided mesh body comprising plural strands, each strand having a first end portion and a second end portion, the plural strands folding over to bring the second end portions of the plural strands adjacent to the first end portions of the plural strands, forming a double-layer of the braided mesh body; and
a pinch member clamping the first end portions and the second end portions of the plural strands, wherein the braided mesh body has an expanded angle ranging from about 30 degrees to about 135 degrees when the braided mesh body is unconstrained in an expanded configuration.

43. The occlusion device of claim 42, wherein the braided mesh body has the expanded angle ranging from about 50 degrees to about 135 degrees when the braided mesh body is unconstrained.

44. The occlusion device of claim 42, wherein the braided mesh body has the expanded angle ranging from about 90 degrees to about 135 degrees when the braided mesh body is unconstrained.

45. The occlusion device of claim 42, wherein the braided mesh body has the expanded angle ranging from about 125 degrees to about 135 degrees when the braided mesh body is unconstrained.

46. The occlusion device of claim 42, wherein the braided mesh body has a maximal width ranging from about 5 mm to about 15 mm when the braided mesh body is unconstrained.

47. The occlusion device of claim 46, wherein the braided mesh body has the expanded angle ranging from about 90 degrees to about 135 degrees.

48. The occlusion device of claim 46, wherein the braided mesh body has the expanded angle ranging from about 125 degrees to about 135 degrees.

49. The occlusion device of claim 42, wherein the braided mesh body has a first expanded angle at a first temperature and second expanded angle at a second temperature.

50. The occlusion device of claim 42, wherein the pinch member is non-radiopaque.

51. The occlusion device of claim 42, wherein the pinch member is constructed from nitinol.

52. The occlusion device of claim 42, wherein the plural strands are constructed from a material comprising a shape-memory material.

53. The occlusion device of claim 42, wherein the braided mesh body or a portion of the mesh body has pores with a pore size ranging from about 20 microns to about 500 microns in the expanded configuration.

54. The occlusion device of claim 42, wherein the braided mesh body or a portion of the mesh body has a porosity ranging from about 5 percent to about 95 percent in the expanded configuration.

55. The occlusion device of claim 42, wherein the braided mesh body or a portion of the mesh body is coated with an anti-thrombogenic material.

56. The occlusion device of claim 42, wherein the braided mesh body or a portion of the mesh body is coated with a material comprising glycosaminoglycan (Heparin) or phosphorylcholines (PC).

57. An occlusion device, comprising:
a braided mesh body comprising plural strands, each strand having a first end portion and a second end portion, the plural strands folding over to bring the second end portions of the plural strands adjacent to the first end portions of the plural strands, forming a double-layer of the braided mesh body; and
at least one pinch member clamping the first end portions and the second end portions of the plural strands, wherein
when the braided mesh body is unconstrained in an expanded configuration state, an inclination of the braided mesh body gradually increases and then gradually decreases from a base end closer to the at least one pinch member to a tail end away from the at least one pinch member thereof, and a ratio of a straight-line distance between the base end and the tail end of a contour line of the braided mesh body to a length of the contour line is greater than or equal to 0.8.

58. The occlusion device of claim 57,
wherein the ratio of the straight-line distance between the base end and the tail end of the contour line of the braided mesh body to the length of the contour line is greater than or equal to 0.9.

59. The occlusion device of claim 57,
wherein there is an intersection point between a connecting line between the base end and the tail end of the contour line and the contour line, and the position of the intersection point in the contour line is defined as a first position, and
wherein a ratio of a width of the first position to a maximal width of the braided mesh body is greater than or equal to 0.5.

60. The occlusion device of claim 57,
wherein an inclination of the braided mesh body at the base end ranges from about 10 degrees to about 40 degrees.

61. The occlusion device of claim 57,
wherein an inclination of the braided mesh body at the tail end ranges from about 15 degrees to about 70 degrees.

62. The occlusion device of claim 57,
wherein the braided mesh body comprises a second position having a maximal inclination between the base end and the tail end, and
wherein the inclination of the second position ranges from about 70 degrees to about 100 degrees.

63. The occlusion device of claim 62,
wherein a ratio of a width of the second position to a maximal width of the braided mesh body is greater than or equal to 0.5.

64. The occlusion device of claim 57,
wherein a maximal width of the braided mesh body ranges from about 5 mm to about 15 mm.

65. The occlusion device of claim 57,
wherein an included angle between the left contour line and the right contour line of the braided mesh body ranges from about 90 degrees to about 170 degrees.
